SEQUENCE LISTING (26 sequences)

SEQ ID NO: 1 - DNA, 480 bp, Porphyromonas gingivalis
atggaaaaaattcccagttttcagttagatcatattcgcctcaaacgaggcatatatgtctcccgcaaggactatatagggggagaggtggttacgactttcgatattcgaatgaaagagcccaatcgcgaaccggtgcttggggcacccgaactgcatacgatcgagcatttggctgcaacttatctgcgtaatcatccgctttataaggacaggatcgttttctgggggccgatgggctgccttacgggcaattactttctgatgcgaggcgattacgtatccaaagatatactgcccctcatgcaggagactttccgcttcatcagagacttcgaaggagaagtgccgggtacggagccgcgcgactgtggcaactgcctgctgcacaacctgccgatggccaaatatgaggccgagaaatacctgcgtgaggtactcgatgtagcgacggaggagaacctgaactatcccgactga

SEQ ID NO: 2 - protein, 159 aa, Porphyromonas gingivalis
MEKIPSFQLDHIRLKRGIYVSRKDYIGGEVVTTFDIRMKEPNREPVLGAPELHTIEHLAATYLRNHPLYKDRIVFWGPMGCLTGNYFLMRGDYVSKDILPLMQETFRFIRDFEGEVPGTEPRDCGNCLLHNLPMAKYEAEKYLREVLDVATEENLNYPD

SEQ ID NO: 3 - DNA, 31 bp, Porphyromonas gingivalis
gtattatcagcggaattcccggcgaaggtcg

SEQ ID NO: 4 - DNA, 32 bp, Porphyromonas gingivalis
gataccgcctccggatccaataatccatccgg

SEQ ID NO: 5 - DNA, 33 bp, Escherichia coli
gcggccgccaccaaatgctcgatcgtatgccag

SEQ ID NO: 6 - DNA, 31 bp, Escherichia coli
tggcggccgcgcgtgaggtactcgatgtagg

SEQ ID NO: 7 - DNA, 25 bp, Porphyromonas gingivalis
agacaatcccgaattcgagatggaa

SEQ ID NO: 8 - DNA, 24 bp, Porphyromonas gingivalis
tgagaaatagagcggatcctaagc

SEQ ID NO: 9 - protein, 60 aa, Borrelia burgorferi
MEKIPSFQLDHIRLKRGIYVSRKDYIGGEVVTTFDIRMKEPNREPVLGAPELHTIEHLAA

SEQ ID NO: 10 - protein, 60 aa, Borrelia burgorferi
TYLRNHPLYKDRIVFWGPMGCLTGNYFLMRGDYVSKDILPLMQETFRFIRDFEGEVPGTE

SEQ ID NO: 11 - protein, 38 aa, Borrelia burgorferi
PRDCGNCLLHNLPMAKYEAEKYLREVLDVATEENLNYP

SEQ ID NO: 12 - protein, 12 aa, Porphyromonas gingivalis
DVYTDHGDLYNT

SEQ ID NO: 13 - protein, 12 aa, Porphyromonas gingivalis
YTPVEEKQNGRM

SEQ ID NO: 14 - protein, 13 aa, Porphyromonas gingivalis
RLSIVPTFGIGKW

SEQ ID NO: 15 - protein, 11 aa, Porphyromonas gingivalis
KWHEPYFGTRL

SEQ ID NO: 16 - protein, 12 aa, Porphyromonas gingivalis
RVVVDNVVYFRI

SEQ ID NO: 17 - protein, 16 aa, Porphyromonas gingivalis
KDDMTGTVNVGLMLKF

SEQ ID NO: 18 - protein, 16 aa, Prophyromonas gingivalis
RNQEINVYNTAEYAKT

SEQ ID NO: 19 - protein, 16 aa, Prophyromonas gingivalis
KGSSEQIYEENAWNRI

SEQ ID NO: 20 - protein, 18 aa, Porphyromonas gingivalis
RLQFTGFDIYGFPQGSKE

SEQ ID NO: 21 - protein, 19 aa, Porphyromonas gingivalis
KIDRNQEINVYNTAEYAKT

SEQ ID NO: 22 - protein, 20 aa, Porphyromonas gingivalis
RRPVSCPECPEPTQPTVTRV

SEQ ID NO: 23 - protein, 16 aa, Porphyromonas gingivalis
KTGTAAYNMKLSERRA

SEQ ID NO: 24 - protein, 385 aa, Porphyromonas gingivalis
MTYRIMKAKSLLLALAGLACTFSATAQEATTQNKAGMHTAFQRDKASDHWFIDIAGGAGMALSGWNNDVDFVDRLSIVPTFGIGKWHEPYFGTRLQFTGFDIYGFPQGSKERNHNYFGNAHLDFMFDLTNYFGVYRPNRVFHIIPWAGIGFGYKFHSENANGEKVGSKDDMTGTVNVGLMLKFRLSRVVDFNIEGQAFAGKMNFIGTKRGKADFPVMATAGLTFNLGKTEWTEIVPMDYALVNDLNNQINSLRGQVEELSRRPVSCPECPEPTQPTVTRVVVDNVVYFRINSAKIDRNQEINVYNTAEYAKTNNAPIKVVGYADEKTGTAAYNMKLSERRAKAVAKMLEKYGVSADRITIEWKGSSEQIYEENAWNRIVVMTAAE

SEQ ID NO: 25 - DNA, 1143 bp, Porphyromonas gingivalis
atgaaagctaaatctttattattagcacttgcgggtctcgcatgcacattcagtgcaacagcccaagaagctactacacagaacaaagcagggatgcacaccgcattccaacgtgataaggcctccgatcattggttcattgacattgcaggtggagcaggtatggctctctcgggatggaataatgatgtagactttgtagatcgtctaagtatcgttcctactttcggtatcggtaaatggcatgagccttatttcggtactcgtctccaattcacaggattcgacatctatggattcccgcaagggagcaaggagcgtaaccacaattactttggaaacgcccaccttgacttcatgttcgatctgacgaactatttcggtgtataccgtcccaatcgtgtcttccatatcatcccatgggcaggtataggatttggttataaattccatagcgaaaacgccaatggtgaaaaagtaggaagtaaagatgatatgaccggaacagttaatgtcggtttgatgctgaaattccgcctatcaagagtcgtagacttcaatattgaaggacaagcttttgccggaaagatgaactttatcgggacaaagagaggaaaagcagacttccctgtaatggctacagcaggtctaacgttcaaccttggcaagacagagtggacagaaattgttcctatggactatgctttggtcaatgacctgaacaaccaaatcaactcacttcgcggtcaagtggaagagttgagccgtcgtcctgtttcatgccctgaatgccctgagcctacacagcctacagttactcgtgtagtcgttgacaatgtggtttacttccgtatcaatagtgcaaagattgatcgtaatcaagaaatcaatgtttacaatacagctgaatatgcgaagaccaacaacgcaccgatcaaggtagtaggttacgctgacgaaaaaaccggtactgcggcctataacatgaagctttcagagcgtcgtgcaaaagcggtagccaagatgcttgaaaagtatggtgtttctgcggatcgcattacaattgaatggaagggctcatcagagcaaatctatgaagagaacgcttggaatcgtattgtagtaatgactgcagcggaataa

SEQ ID NO: 26 - protein, 380 aa, Porphyromonas gingivalis
MKAKSLLLALAGLACTFSATAQEATTQNKAGMHTAFQRDKASDHWFIDIAGGAGMALSGWNNDVDFVDRLSIVPTFGIGKWHEPYFGTRLQFTGFDIYGFPQGSKERNHNYFGNAHLDFMFDLTNYFGVYRPNRVFHIIPWAGIGFGYKFHSENANGEKVGSKDDMTGTVNVGLMLKFRLSRVVDFNIEGQAFAGKMNFIGTKRGKADFPVMATAGLTFNLGKTEWTEIVPMDYALVNDLNNQINSLRGQVEELSRRPVSCPECPEPTQPTVTRVVVDNVVYFRINSAKIDRNQEINVYNTAEYAKTNNAPIKVVGYADEKTGTAAYNMKLSERRAKAVAKMLEKYGVSADRITIEWKGSSEQIYEENAWNRIVVMTAAE

(19) United States
(12) Patent Application Publication
(10) Pub. No.: US 2003/0083287 A1
(43) Pub. Date: May 1, 2003

(54) ginS

(76) Inventors: Nicola A. Burgess, Lichfield (GB); Miguel M. Camara Garcia, Chesterfield (GB); David F. Kirke, Kimberley (GB); Nicholas L. Meyers, Huntingdon (GB); Paul Williams, Kimberley (GB)

Correspondence Address: Edward R. Gimmi, SmithKline Beecham Corporation, Corporate Intellectual Property -U.S., UW2220, P.O. Box 1539, King of Prussia, PA, 19406-0939, US

(21) Appl. No.: 09/998,279

(22) Filed: Nov. 30, 2001

Related U.S. Application Data
(60) Provisional application No. 60/250,288, filed on Nov. 30, 2000.

(51) Int. Cl. A61K 48/00; A61K 38/16; C12P 21/02; C12N 1/21; C12N 15/74; C07H 21/04

(52) U.S. Cl. 514/44; 514/12; 530/350; 536/23.7; 435/69.1; 435/252.3; 435/320.1

(57) ABSTRACT

The invention provides ginS polypeptides and, polynucleotides encoding ginS polypeptides and methods for producing such polypeptides by recombinant techniques. Also provided are methods for utilizing ginS polypeptides to screen for antibacterial compounds.

### FIELD OF THE INVENTION

This invention relates to newly identified polynucleotides and polypeptides, and their production and uses, as well as their variants, agonists and antagonists, and their uses. In particular, the invention relates to polynucleotides and polypeptides of the ginS (autoinducer synthesis) family, as well as their variants, herein referred to as “ginS,” “ginS polynucleotide(s),” and “ginS polypeptide(s)” as the case may be.

### BACKGROUND OF THE INVENTION

It is particularly preferred to employ genes and gene products from Porphyromonas gingivalis as targets for the development of antibiotics. Porphyromonas gingivalis is a clinically important microorgansim found within the human oral cavity gingival within crevicular fluid, as a member of the subgingival plaque, on the tongue, tonsils, and pharynx (Mayrand, et al., Micro. Rev. 52: 134-152 (1988). Porphyromonas gingivalis is a non-motile, Gram negative anaerobic bacterium involved in the generation of breath malodour and strongly implicated in causing periodontal disease in humans. Recent evidence also demonstrates a clear correlation between systemic exposure to Porphyromonas gingivalis with cardiovascular disease, diabetes and an increased likelihood of pre-term births (up to a 7× risk increase for spontaneous pre-term births, possibly caused by by-products of bacterial metabolism which induce the release of mediators linked to the initiation of labour) and low neonatal birth weight (18% of low birth weights may be attributable to periodonitis) (Slots, et al. J. Clin. Periodontol. 13: 570-577 (1986); Maiden, et al., J. Clin. Periodontol. 17: 1-13 (1990); Offenbacher, et al., J. Periodontol. 67: 1103-1113 (1996)). Porphyromonas gingivalis elaborates a large number of virulence factors including fimbriae (Lamont, et al., Oral Micro. Immunol. 8: 272-276 (1993); Miyata, et al., Infect. Immun. 65: 3515-3519 (1997); Pike, et al.,. J. Bacteriol. 178: 2876-2882 (1996); Weinberg, et al., Infect. Immun. 65: 313-316 (1997); Yoneda, et al. Oral Microbiol. Immunol. 11: 129-134 (1996)) and two major classes of proteases, KGP (Lys-X gingipain) and RGP (Arg-X gingipain) (Kuramitsu, et al., J. Periodontol. Res. 32: 140-142 (1997); Nakayama, et al., J. Biol. Chem. 270: 23619-23626 (1995); Okamoto, et al., J. Biochem. 120: 398-406 (1996); Park, et al., Infect. Immun. 61: 4139-4146 (1993)), known to be involved in mediating cellular attachment, tissue invasion, destruction (Birkedal-Hansen, et al., J Periodontal Res. 23: 258-64 (1988); Kato, et al., J. Bacteriol. 174: 3889-3895 (1992); Lantz, et al., J. Bacteriol. 173: 495-504 (1991)) and immune evasion (Imamura, et al., J. Clin. Invest. 94; 361-367 (1994); Nilsson, et al., Infect. Immun. 50: 467-471 (1985)). It has been shown in several other bacteria including Aeromonas hydrophila (Swift, et al., TIBS 21: 214-219 (1996). Envinia carotovora (Bainton, et al., Gene, 116: 87-91 (1992); Jones, et al., EMBO J. 12: 2477-2482 (1993)) and Pseudomonas aeruginosa (Gambello, et al., J. Bacteriol. 173: 3000-3009 (1991); Latifi, et al., Mol. Micro. 17: 333-343 (1995); Pearson, et al., J. Bacteriol. 179: 5756-5767 (1997)), that extracellular protease activity is controlled by the cell density-dependent system known as quorum sensing. Many bacteria use a mechanism homologous to that of Vibrio fischeri in which the signalling molecule (or autoinducer) is an N-acylhomoserine lactone (AHL) synthesized by luxI and responded to by direct binding to the transcriptional regulator luxR (Williams, et al., FEMS Micro. Lett. 100: 161-168 (1992); Salmond, et al., Mol. Microbiol. 16: 615-624 (1995); Swift, et al., Berlin Heidelberg: Springer-Verlag, pp.185-207 (1998); Stevens, et al., J. Bacteriol. 179: 557-562 (1997)). A diverse number of phenotypes are regulated in this way, including bioluminescence, motility and expression of virulence determinants (Bainton, et al., Biochem. J. 288: 997-1004 (1992); Bainton, et al., Gene, 116: 87-91 (1992).

In the bioluminescent bacterium Vibrio harveyi, two independent quorum sensing systems are found which are controlled by a number of proteins, none of which are homologous to the luxR/I proteins from Vibrio fischeri (Cao, et al., J. Bacteriol. 175: 3856-3862 (1993), Bassler, et al., Mol. Micro. 12: 403-412 (1994a), Bassler, et al., Mol. Micro. 13: 273-286 (1994b), Freeman, et al., Mol. Micro. 31: 665-677 (1999a), Freeman, et al., J. Bacteriol. 181: 899-906 (1999). One system, however, does involve the synthesis of an AHL (3-OH-C4-HSL) by the signal generators luxLM. Once a critical concentration is reached, 3-OH-C4-HSL interacts with the response regulator luxN. The second system requires luxS, homologues of which were identified in a number of bacteria including Escherichia coli, Salmonella typhimurium, Bacillus subtilis and Helicobacter pylori (Surette, et al., Proc. Natl. Acad. Sci, USA. 96: 1639-1644 (1999a), Surette, et al., Mol. Micro. 31: 585-595 (1999b), Joyce, et al., J. Bacteriol. 182: 3638-3643 (2000).

LuxS is involved in the production of a signalling molecule of unknown chemical structure, AI-2, which interacts with response regulators, luxPQ. Information from both quorum sensing systems is relayed to the two-component regulator luxO, via a phospho-relay protein, luxU. Dephosphorylation of luxO results in activation of luminescence (Freeman, et al., Mol. Micro. 31: 665-677 (1999a), Freeman, et al., J. Bacteriol. 181: 899-906 (1999b).

It is thought that luxS is involved in bacterial cross-talk since it is present in both Gram-positive and Gram-negative bacteria (Surette, et al., Proc. Natl. Acad. Sci, USA. 96: 1639-1644 (1999a), Surette, et al.,. Mol. Micro. 31: 585-595 (1999b), and it has been reported to influence type III secretion in enterovirulent Escherichia coli (Sperandio, et al., PNAS 96: 15196-15201 (1999).

Breakdown of tissue proteins is an essential feature of the pathogenesis of periodontal disease and similarly, protein breakdown of dietary proteins is a critical step leading to breath malodour. Bacteria such as Porphyromonas gingivalis and Prevotella intermedia must break down host gingival connective tissue in order to replicate and cause disease; breakdown products from protein include volatile sulphur compounds, principally H₂S and methylmercaptan and also diamines such as putrescine and cadaverine, which are among the main causes of bad breath. Many of the important odourigenic anaerobes present in the oral cavity e.g, Fusobacterium nucleatum, Haemophilus segnis, Haemophilus parainfluenzae, Veillonella alcalescens and Veillonella parvula generate odorous compounds when supplied with a free amino acids e.g, cysteine, methionine, tryptophan, omithine and arginine, but are themselves unable to break down longer chain polypeptides. It has been proposed that the proteolytic activity of Porphyromonas gingivalis, particularly dipeptidyl peptidase and deaminase activity, is central to the ability of this organism to break down dietary proteins, which then liberate free amino-acids subsequently metabolised by other plaque bacteria resulting in the development of breath malodour.

Because of the difficulty in mechanically removing the anaerobic plaque from interdental areas and from the tongue dorsum and also, because of the predisposition of chlorhexidine (the active ingredient found in mouthrinses commonly employed to treat gingivitis and restore oral hygiene) to be bitter-tasting and to cause extrinsic staining of teeth, there is a clear unment clinical need for polynucleotides and polypeptides, such as the ginS embodiments of the invention, that have a present benefit of, among other things, being useful to screen compounds for antimicrobial activity and/or the ability to inhibit the proteolytic and/or deaminative activity associated with this organism which could be developed as dentifrices, mouthrinses, dental lozenges and/or dental gums.

Although many different bacterial genera have been found to be associated with periodontal disease, Porphyromonas gingivalis is now known to be one of the critical bacteria species involved in the progression of gingivitis and the invasive infection of the host tissue arising from the periodontal pocket, largely because of its' significant proteolytic activity.

Several forms of periodontal disease are now recognised, based upon the presence or absence of inflammation, extent and pattern of attachment loss, probing pocket depth, patient's age at onset, rate of progression, and presence of various signs and symptoms e.g, pain and ulceration. However, all share key common features in that the disease affects the periodontium or supporting structures of the teeth including gingiva, periodontal ligament, and alveolar bone. Bacterial infection is the primary etiological factor causing periodontal disease, and the majority of these diseases are inflammatory lesions caused by the accumulation of microorganisms around the gingival margin.

Gingivitis defines inflammation that is confined to the gingiva, while periodontitis is characterized by subsequent destruction of bone and periodontal ligament resulting in loss of attachment to the tooth. Periodontitis is defined as a chronic inflammatory disease of the periodontium occurring in response to bacterial plaque on the adjacent teeth; characterized by gingivitis, destruction of the alveolar bone and periodontal ligament, apical migration of the epithelial attachment resulting in the formation of periodontal pockets, and ultimately loosening and exfoliation of the teeth.

One hypothesis is that virulence gene expression in Porphyromonas gingivalis may be regulated via the production of molecules related to the AHL/Lux quorum sensing system, employed by several species of non-oral Gram -ve bacteria. To date, no homologues of AHL-based signalling system have been identified in oral bacteria. However, gins was first identified by homology with the luxS gene of Vibrio harveyi which encodes a soluble signalling molecule, AI-2.

Periodontal disease is currently by surgical excision of diseased tissue either alone or in combination with systemic or locally acting antibitoics, particularly the tetracyclines. In severe cases, this may need to be followed by reconstructive surgery. However, given the concern surrounding the use of antibitoics and the generation of antibiotic-resistant clinical isolates, the issues surrounding the use of tetratcyclines in children (tetracyclines become incorporated into developing teeth and bones) and the difficulties in delivering and maintaining effective drug levels locally e.g, from polymeric chips or surgically-implanted sutures, it is clear that there is an unmet medical need and demand for new anti-microbial agents, vaccines, drug screening methods, and diagnostic tests targeted at Porphyromonas gingivalis.

Moreover, the drug discovery process is currently undergoing a fundamental revolution as it embraces “functional genomics,” that is, high throughput genome- or gene-based biology. This approach is rapidly superseding earlier approaches based on “positional cloning” and other methods. Functional genomics relies heavily on the various tools of bioinformatics to identify gene sequences of potential interest from the many molecular biology databases now available as well as from other sources. There is a continuing and significant need to identify and characterize further genes and other polynucleotides sequences and their related polypeptides, as targets for drug discovery.

Clearly, there exists a need for polynucleotides and polypeptides, such as the ginS embodiments of the invention, that have a present benefit of, among other things, being useful to screen compounds for antimicrobial activity. Such factors are also useful to determine their role in pathogenesis of infection, dysfunction and disease. There is also a need for identification and characterization of such factors and their antagonists and agonists to find ways to prevent, ameliorate or correct such infection, dysfunction and disease.

### SUMMARY OF THE INVENTION

The present invention relates to ginS, in particular ginS polypeptides and ginS polynucleotides, recombinant materials and methods for their production. In another aspect, the invention relates to methods for using such polypeptides and polynucleotides, including treatment of microbial diseases, amongst others. In a further aspect, the invention relates to methods for identifying agonists and antagonists using the materials provided by the invention, and for treating microbial infections and conditions associated with such infections with the identified agonist or antagonist compounds. In a still further aspect, the invention relates to diagnostic assays for detecting diseases associated with microbial infections and conditions associated with such infections, such as assays for detecting ginS expression or activity.

Various changes and modifications within the spirit and scope of the disclosed invention will become readily apparent to those skilled in the art from reading the following descriptions and from reading the other parts of the present disclosure.

### BRIEF DESCRIPTION OF THE FIGURES

FIG. 1 shows V.harveyi bioluminescence assay demonstrating complementation of E.coli DH5α luxS mutation by pMALGin1. FIG. 1 further shows the V.harveyi luminescence assay uses overnight culture supernatants demonstrating complementation of the E.coli DH5α luxS_{EC} mutation in DH5α expressing gins (GinS1 & GinS2) and reduced bioluminescence of two ginS⁻ null mutants (Mut1 & Mut2). Negative control=E.coli DH5α; Positive control=V.harveyi BB120.

FIG. 2 shows the same data exhibited in FIG. 1 expressed as a histogram.

FIG. 3 shows SDS-PAGE analysis demonstrating expression of MalE-GinS protein fusion. Further, FIG. 3 shows SDS-PAGE analysis showing the expressed MalE-GinS protein fusion. pMAL-c2 was induced with 0.3 mM IPTG. Lane 1 shows DH5-α; lane 2 MalE; lane 3 MalE-GinS. Lanes 1-3 are soluble; lanes 4-6 are the same as 1-3 but are insoluble fractions.

FIG. 4 shows MalE-GinS fusion construct induces expression of biologically active AI-2 in E.coli DH5α, dompelementing the mutation in this strain

FIG. 5 shows purification of the MalE-GinS fusion protein by affinity chromatography.

FIG. 6 shows SDS-PAGE analysis of fractions collected from affinity chromotography column.

FIG. 7 shows SDS-PAGE analysis showing Factor Xa cleavage of GinS from the MalE fusion protein. Further, FIG. 7 shows SDS-PAGE analysis showing cleavage of GinS from MalE following incubation with Factor Xa at RT. Lanes 1 & 2 show uncut fusion; lane 3: 2 hours incubation; lane 4: 4 hours incubation; lane 5: 6 hours incubation; lane 6: 22 hours incubation.

FIG. 8 shows production of AI-2 and MalE-GinS throughout the growth cycle in E.coli This data demonstrates that AI-2 production peaks between 7 and 9 hours of the growth curve. This coincides with gins expression (see Western blot inset), which appears to be maximal by 5 hours and is maintained through to 24 hours of the growth curve. This temporal link would fit with the model that ginS is required for expression of AI-2.

FIG. 9 shows production of AI-2 and expression of MalE-GinS throughout growth of P. gingivalis. Western blots were carried out using a polyclonal antibody to probe GinS from cell lysates of the wild-type P.gingivalis W50 (Panel A) and the ginS⁻ null mutant (Panel B) throughout the growth curve. Lane 1 show Positive GinS control; Lanes 2-7 show cell lysates at 6, 12, 24, 72 & 96 hours of growth.

FIG. 10 shows confirmation of creation of insertional ‘Null’ mutation in ginS in P.gingivalis W50 by Agarose Gel Electrophoretic Analysis of PCR Amplification of erm Cassette.

FIG. 11 shows Southern blot analysis demonstrating chromosomal integration of the erm Cassette in the P.gingivalis ginS⁻ Null Mutant. The control (lane 1) demonstrates hybridization of probe to the ginS gene amplified from a plasmid. Lanes 2 & 3 demonstrate hybridization to ginS sequences amplified from the P.gingivalis chromosome. Prior to Southern blotting, DNA was cut with a restriction enzyme with two cleavage sites within ginS. This results in two hybridizing bands in the wild-type but only a single in the ginS⁻ null mutant, due to elimination of the internal restriction site following replacement with the erm cassette.

FIG. 12 shows comparison of Total Protease Expression in Wild-type and ginS⁻ Null Mutant Culture Supernatants by Zymography

FIG. 13 shows Expression of Kgp (Lys-X gingipain) and RgpA (Arg-X gingipain) Proteases in Wildtype P.gingivalis Strain W50 Compared to the ginS⁻ Mutant by SDS-PAGE Analysis of Total Protein Lysates, Probed with Specific Antibodies to Kgp & RgpA.

FIG. 14 shows determination of KGP and RGP Protease Activity in Whole Cell Lysates and Culture Supernatants of P.gingivalis W50 and the ginS⁻ Null Mutant.

FIG. 15 shows Total Rgp (Arg-X Gingipain) Activity in Soluble Cell Fractions of Wildtype P.gingivalis and the ginS⁻ Null Mutant Over Time.

FIG. 16 shows total Rgp (Arg-X Gingipain) Activity in Culture Supernatants from Wildtype P.gingivalis and the ginS⁻ Null Mutant Over Time.

FIG. 17 shows determination of Haemagglutinin Activity in ginS⁻ Null Mutant. In accordance with the direct structural relationship of proteases and haemagglutinins in P.gingivalis (Yoneda et al, 1996), a 4-fold decrease in agglutination of sheep erythrocytes was observed with the ginS⁻ mutant of P.gingivalis, further confirming a decrease in production of proteases.

FIG. 18 shows 2-D Gel Electrophoresis of Total Protein Lysates from Wild-type P.gingivalis W50 and ginS⁻ Null Mutant

### DESCRIPTION OF THE INVENTION

The invention relates to ginS polypeptides and polynucleotides as described in greater detail below. In particular, the invention relates to polypeptides and polynucleotides of ginS from Porphyromonas gingivalis, that is related by amino-acid sequence homology to the luxS polypeptide of Borrelia Bergdorferi (ATCC 35210). The invention relates especially to ginS having a nucleotide and amino-acid sequences set out in Table 1 as SEQ ID NO:1 and SEQ ID NO:2 respectively. Note that sequences recited in the Sequence Listing below as “DNA” represent an exemplification of the invention, since those of ordinary skill will recognize that such sequences can be usefully employed in polynucleotides in general, including ribopolynucleotides.

TABLE 1
GinS Polynucleotide and ginS Polypeptide Sequences

(A) Porphyromonas gingivalis W50 ginS polynucleo-
tide coding sequence.

[SEQ ID NO:1]
5′-ATGGAAAAAATTCCCAGTTTTCAGTTAGATCATATTCGCCTCAAACG
AGGCATATATGTCTCCCGCAAGGACTATATAGGGGGAGAGGTGGTTACGA
CTTTCGATATTCGAATGAAAGAGCCCAATCGCGAACCGGTGCTTGGGGCA
CCCGAACTGCATACGATCGAGCATTTGGCTGCAACTTATCTGCGTAATCA
TCCGCTTTATAAGGACAGGATCGTTTTCTGGGGGCCGATGGGCTGCCTTA
CGGGCAATTACTTTCTGATGCGAGGCGATTACGTATCCAAAGATATACTG
CCCCTCATGCAGGAGACTTTCCGCTTCATCAGAGACTTCGAAGGAGAAGT
GCCGGGTACGGAGCCGCGCGACTGTGGCAACTGCCTGCTGCACAACCTGC
CGATGGCCAAATATGAGGCCGAGAAATACCTGCGTGAGGTACTCGATGTA
GCGACGGAGGAGAACCTGAACTATCCCGACTGA-3′
(B) Porphyromonas gingivalis W50 ginS polypeptide
sequence deduced from a polynucleotide sequence in
this table.
[SEQ ID NO:2]
NH₂-MEKIPSFQLDHIRLKRGIYVSRKDYIGGEVVTTFDIRMKEPNREPV
LGAPELHTIEHLAATYLRNHPLYKDRIVFWGPMGCLTGNYFLMRGDYVSK
DILPLMQETFRFIRDFEGEVPGTEPRDCGNCLLHNLPMAKYEAEKYLREV
LDVATEENLNYPD.-COOH
Origination of Materials

Porphyromonas gingivalis strain W50 (Shah, et al., Oral Microbiol Immunol 4:19-23 (1989) comprises a full length ginS gene. The sequence of the polynucleotides comprised in this strain, as well as the amino acid sequence of any polypeptide encoded thereby, are controlling in the event of any conflict with any description of sequences herein.

In one aspect of the invention there is provided an isolated nucleic acid molecule encoding a mature polypeptide expressible by the Porphyromonas gingivalis W50 strain, which polypeptide is comprised in this original strain strain. Further provided by the invention are ginS polynucleotide sequences in the original strain, such as DNA and RNA, and amino acid sequences encoded thereby. Also provided by the invention are ginS polypeptide and polynucleotide sequences isolated from the original strain.

Polypeptides

ginS polypeptide of the invention is substantially phylogenetically related to other proteins of the luxS (autoinducer synthesis) family.

In one aspect of the invention there are provided polypeptides of Porphyromonas gingivalis referred to herein as “ginS” and “ginS polypeptides” as well as biologically, diagnostically, prophylactically, clinically or therapeutically useful variants thereof, and compositions comprising the same.

Among the particularly preferred embodiments of the invention are variants of ginS polypeptide encoded by naturally occurring alleles of a ginS gene.

The present invention further provides for an isolated polypeptide that: (a) comprises or consists of an amino acid sequence that has at least 95% identity, most preferably at least 97-99% or exact identity, to that of SEQ ID NO:2 over the entire length of SEQ ID NO:2; (b) a polypeptide encoded by an isolated polynucleotide comprising or consisting of a polynucleotide sequence that has at least 95% identity, even more preferably at least 97-99% or exact identity to SEQ ID NO:1 over the entire length of SEQ ID NO:1; (c) a polypeptide encoded by an isolated polynucleotide comprising or consisting of a polynucleotide sequence encoding a polypeptide that has at least 95% identity, even more preferably at least 97-99% or exact identity, to the amino acid sequence of SEQ ID NO:2, over the entire length of SEQ ID NO:2.

The polypeptides of the invention include a polypeptide of Table 1 [SEQ ID NO:2] (in particular a mature polypeptide) as well as polypeptides and fragments, particularly those that has a biological activity of ginS, and also those that have at least 95% identity to a polypeptide of Table 1 [SEQ ID NO:2] and also include portions of such polypeptides with such portion of the polypeptide generally comprising at least 30 amino-acids and more preferably at least 50 amino-acids.

The invention also includes a polypeptide consisting of or comprising a polypeptide of the formula:

X—(R₁)ₘ—(R₂)—(R₃)ₙ—Y

wherein, at the amino terminus, X is hydrogen, a metal or any other moiety described herein for modified polypeptides, and at the carboxyl terminus, Y is hydrogen, a metal or any other moiety described herein for modified polypeptides, R₁ and R₃ are any amino acid residue or modified amino acid residue, m is an integer between 1 and 1000 or zero, n is an integer between 1 and 1000 or zero, and R₂ is an amino acid sequence of the invention, particularly an amino acid sequence selected from Table 1 or modified forms thereof. In the formula above, R₂ is oriented so that its amino terminal amino acid residue is at the left, covalently bound to R₁, and its carboxy terminal amino acid residue is at the right, covalently bound to R₃. Any stretch of amino acid residues denoted by either R₁ or R₃, where m and/or n is greater than 1, may be either a heteropolymer or a homopolymer, preferably a heteropolymer. Other preferred embodiments of the invention are provided where m is an integer between 1 and 50, 100 or 500, and n is an integer between I and 50, 100, or 500.

It is most preferred that a polypeptide of the invention is derived from Porphyromonas gingivalis, however, it may preferably be obtained from other organisms of the same taxonomic genus. A polypeptide of the invention may also be obtained, for example, from organisms of the same taxonomic family or order.

A fragment is a variant polypeptide having an amino acid sequence that is entirely the same as part but not all of any amino acid sequence of any polypeptide of the invention. As with ginS polypeptides, fragments may be “free-standing,” or comprised within a larger polypeptide of which they form a part or region, most preferably as a single continuous region in a single larger polypeptide.

Preferred fragments include, for example, truncation polypeptides having a portion of an amino acid sequence of Table 1 [SEQ ID NO:2], or of variants thereof, such as a continuous series of residues that includes an amino- and/or carboxyl-terminal amino acid sequence. Degradation forms of the polypeptides of the invention produced by or in a host cell, particularly a Porphyromonas gingivalis, are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

Further preferred fragments include an isolated polypeptide comprising an amino acid sequence having at least 15, 20, 30, 40, 50 or 100 contiguous amino acids from the amino acid sequence of SEQ ID NO:2, or an isolated polypeptide comprising an amino acid sequence having at least 15, 20, 30, 40, 50 or 100 contiguous amino acids truncated or deleted from the amino acid sequence of SEQ ID NO:2.

Fragments of the polypeptides of the invention may be employed for producing the corresponding full-length polypeptide by peptide synthesis; therefore, these variants may be employed as intermediates for producing the full-length polypeptides of the invention.

Polynucleotides

It is an object of the invention to provide polynucleotides that encode ginS polypeptides, particularly polynucleotides that encode a polypeptide herein designated ginS.

In a particularly preferred embodiment of the invention the polynucleotide comprises a region encoding ginS polypeptides comprising a sequence set out in Table 1 [SEQ ID NO:1] that includes a full length gene, or a variant thereof. The Applicants believe that this full length gene is essential to the growth and/or survival of an organism that possesses it, such as Porphyromonas gingivalis.

As a further aspect of the invention there are provided isolated nucleic acid molecules encoding and/or expressing ginS polypeptides and polynucleotides, particularly Porphyromonas gingivalis ginS polypeptides and polynucleotides, including, for example, unprocessed RNAs, nrbozyme RNAs, mRNAs, cDNAs, genomic DNAs, B- and Z-DNAs. Further embodiments of the invention include biologically, diagnostically, prophylactically, clinically or therapeutically useful polynucleotides and polypeptides, and variants thereof, and compositions comprising the same.

Another aspect of the invention relates to isolated polynucleotides, including at least one full length gene, that encodes a ginS polypeptide having a deduced amino-acid sequence of Table 1 [SEQ ID NO:2] and polynucleotides closely related thereto and variants thereof.

In another particularly preferred embodiment of the invention there is a ginS polypeptide from Porphyromonas gingivalis comprising or consisting of an amino-acid sequence of Table 1 [SEQ ID NO:2], or a variant thereof.

Using the information provided herein, such as a polynucleotide sequence set out in Table 1 [SEQ ID NO:1], a polynucleotide of the invention encoding ginS polypeptide may be obtained using standard cloning and screening methods, such as those for cloning and sequencing chromosomal DNA fragments from bacteria using Porphyromonas gingivalis W50 cells as starting material, followed by obtaining a full length clone. For example, to obtain a polynucleotide sequence of the invention, such as a polynucleotide sequence given in Table 1 [SEQ ID NO:1], typically a library of clones of chromosomal DNA of Porphyromonas gingivalis W50 in Escherichia coli or some other suitable host is probed with a radiolabeled oligonucleotide, preferably a 17-mer or longer, derived from a partial sequence. Clones carrying DNA identical to that of the probe can then be distinguished using stringent hybridization conditions. By sequencing the individual clones thus identified by hybridization with sequencing primers designed from the original polypeptide or polynucleotide sequence it is then possible to extend the polynucleotide sequence in both directions to determine a full length gene sequence. Conveniently, such sequencing is performed, for example, using denatured double stranded DNA prepared from a plasmid clone. Suitable techniques are described by Maniatis, T., Fritsch, E. F. and Sambrook et al., Molecular Cloning: A Laboratory Manual, 2nd Ed.; Cold Spring Harbor Laboratory Press, Cold Spring Harbor, N.Y. (1989). (see in particular Screening By Hybridization 1.90 and Sequencing Denatured Double-Stranded DNA Templates 13.70). Direct genomic DNA sequencing may also be performed to obtain a full length gene sequence. Illustrative of the invention, each polynucleotide set out in Table 1 [SEQ ID NO: 1] was discovered in a DNA library derived from Porphyromonas gingivalis.

Moreover, each DNA sequence set out in Table 1 [SEQ ID NO:1] contains an open reading frame encoding a protein having about the number of amino acid residues set forth in Table 1 [SEQ ID NO:2] with a deduced molecular weight that can be calculated using amino-acid residue molecular weight values well known to those skilled in the art. The polynucleotide of SEQ ID NO:1, between nucleotide number 1 and the stop codon that begins at nucleotide number 469 of SEQ ID NO:1, encodes the polypeptide of SEQ ID NO:2.

In a further aspect, the present invention provides for an isolated polynucleotide comprising or consisting of: (a) a polynucleotide sequence that has at least 95% identity, even more preferably at least 97-99% or exact identity to SEQ ID NO:1 over the entire length of SEQ ID NO:1; (b) a polynucleotide sequence encoding a polypeptide that has at least 95% identity, even more preferably at least 97-99% or 100% exact, to the amino acid sequence of SEQ ID NO:2, over the entire length of SEQ ID NO:2.

A polynucleotide encoding a polypeptide of the present invention, including homologs and orthologs from species other than Porphyromonas gingivalis, may be obtained by a process that comprises the steps of screening an appropriate library under stringent hybridization conditions with a labeled or detectable probe consisting of or comprising the sequence of SEQ ID NO:1 or a fragment thereof; and isolating a full-length gene and/or genomic clones comprising said polynucleotide sequence.

The invention provides a polynucleotide sequence identical over its entire length to a coding sequence (open reading frame) in Table 1 [SEQ ID NO:1]. Also provided by the invention is a coding sequence for a mature polypeptide or a fragment thereof, by itself as well as a coding sequence for a mature polypeptide or a fragment in reading frame with another coding sequence, such as a sequence encoding a leader or secretory sequence, a pre-, or pro- or prepro-protein sequence. The polynucleotide of the invention may also comprise at least one non-coding sequence, including for example, but not limited to at least one non-coding 5′ and 3′ sequence, such as the transcribed but non-translated sequences, termination signals (such as rho-dependent and rho-independent termination signals), ribosome binding sites, Kozak sequences, sequences that stabilize mRNA, introns, and polyadenylation signals. The polynucleotide sequence may also comprise additional coding sequence encoding additional amino acids. For example, a marker sequence that facilitates purification of a fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA peptide tag (Wilson et al., Cell 37: 767 (1984), both of that may be useful in purifying polypeptide sequence fused to them. Polynucleotides of the invention also include, but are not limited to, polynucleotides comprising a structural gene and its naturally associated sequences that control gene expression.

A preferred embodiment of the invention is a polynucleotide of consisting of or comprising nucleotide 1 to the nucleotide immediately upstream of or including nucleotide 469 set forth in SEQ ID NO:1 of Table 1, both of that encode a ginS polypeptide.

The invention also includes a polynucleotide consisting of or comprising a polynucleotide of the formula:

X—(R₁)ₘ—(R₂)—(R₃)ₙ—Y

wherein, at the 5′ end of the molecule, X is hydrogen, a metal or a modified nucleotide residue, or together with Y defines a covalent bond, and at the 3′ end of the molecule, Y is hydrogen, a metal, or a modified nucleotide residue, or together with X defines the covalent bond, each occurrence of R₁ and R₃ is independently any nucleic acid residue or modified nucleic acid residue, m is an integer between 1 and 3000 or zero, n is an integer between 1 and 3000 or zero, and R₂ is a nucleic acid sequence or modified nucleic acid sequence of the invention, particularly a nucleic acid sequence selected from Table 1 or a modified nucleic acid sequence thereof. In the polynucleotide formula above, R₂ is oriented so that its 5′ end nucleic acid residue is at the left, bound to R₁, and its 3′ end nucleic acid residue is at the right, bound to R₃. Any stretch of nucleic acid residues denoted by either R₁ and/or R₂, where m and/or n is greater than 1, may be either a heteropolymer or a homopolymer, preferably a heteropolymer. Where, in a preferred embodiment, X and Y together define a covalent bond, the polynucleotide of the above formula is a closed, circular polynucleotide, that can be a double-stranded polynucleotide wherein the formula shows a first strand to which the second strand is complementary. In another preferred embodiment m and/or n is an integer between 1 and 1000. Other preferred embodiments of the invention are provided where m is an integer between 1 and 50, 100 or 500, and n is an integer between 1 and 50, 100, or 500.

It is most preferred that a polynucleotide of the invention is derived from Porphyromonas gingivalis, however, it may preferably be obtained from other organisms of the same taxonomic genus. A polynucleotide of the invention may also be obtained, for example, from organisms of the same taxonomic family or order.

The term “polynucleotide encoding a polypeptide” as used herein encompasses polynucleotides that include a sequence encoding a polypeptide of the invention, particularly a bacterial polypeptide and more particularly a polypeptide of the Porphyromonas gingivalis ginS having an amino-acid sequence set out in Table 1 [SEQ ID NO:2]. The term also encompasses polynucleotides that include a single continuous region or discontinuous regions encoding the polypeptide (for example, polynucleotides interrupted by integrated phage, an integrated insertion sequence, an integrated vector sequence, an integrated transposon sequence, or due to RNA editing or genomic DNA reorganization) together with additional regions, that also may comprise coding and/or non-coding sequences.

The invention further relates to variants of the polynucleotides described herein that encode variants of a polypeptide having a deduced amino acid sequence of Table 1 [SEQ ID NO:2]. Fragments of polynucleotides of the invention may be used, for example, to synthesize full-length polynucleotides of the invention.

Further particularly preferred embodiments are polynucleotides encoding ginS variants, that have the amino acid sequence of ginS polypeptide of Table 1 [SEQ ID NO:2] in which several, a few, 5 to 10, 1 to 5, 1 to 3, 2, 1 or no amino acid residues are substituted, modified, deleted and/or addes, in any combination. Especially preferred among these are silent substitutions, additions and deletions, that do not alter the properties and activities of ginS polypeptide.

Preferred isolated polynucleotide embodiments also include polynucleotide fragments, such as a polynucleotide comprising a nuclic acid sequence having at least 15, 20, 30, 40, 50 or 100 contiguous nucleic acids from the polynucleotide sequence of SEQ ID NO:1, or an polynucleotide comprising a nucleic acid sequence having at least 15, 20, 30, 40, 50 or 100 contiguous nucleic acids truncated or deleted from the 5′ and/or 3′ end of the polynucleotide sequence of SEQ ID NO:1.

Further preferred embodiments of the invention are polynucleotides that are at least 95% or 97% identical over their entire length to a polynucleotide encoding ginS polypeptide having an amino acid sequence set out in Table 1 [SEQ ID NO:2], and polynucleotides that are complementary to such polynucleotides. Most highly preferred are polynucleotides that comprise a region that is at least 95% are especially preferred. Furthermore, those with at least 97% are highly preferred among those with at least 95%, and among these those with at least 98% and at least 99% are particularly highly preferred, with at least 99% being the more preferred.

Preferred embodiments are polynucleotides encoding polypeptides that retain substantially the same biological function or activity as a mature polypeptide encoded by a DNA of Table 1 [SEQ ID NO:1].

In accordance with certain preferred embodiments of this invention there are provided polynucleotides that hybridize, particularly under stringent conditions, to ginS polynucleotide sequences, such as those polynucleotides in Table 1.

The invention further relates to polynucleotides that hybridize to the polynucleotide sequences provided herein. In this regard, the invention especially relates to polynucleotides that hybridize under stringent conditions to the polynucleotides described herein. As herein used, the terms “stringent conditions” and “stringent hybridization conditions” mean hybridization occurring only if there is at least 95% and preferably at least 97% identity between the sequences. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 533 Denhardt's solution, 10% dextran sulfate, and 20 micrograms/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein. Solution hybridization may also be used with the polynucleotide sequences provided by the invention.

The invention also provides a polynucleotide consisting of or comprising a polynucleotide sequence obtained by screening an appropriate library comprising a complete gene for a polynucleotide sequence set forth in SEQ ID NO:1 under stringent hybridization conditions with a probe having the sequence of said polynucleotide sequence set forth in SEQ ID NO:1 or a fragment thereof; and isolating said polynucleotide sequence. Fragments useful for obtaining such a polynucleotide include, for example, probes and primers fully described elsewhere herein.

As discussed elsewhere herein regarding polynucleotide assays of the invention, for instance, the polynucleotides of the invention, may be used as a hybridization probe for RNA, cDNA and genomic DNA to isolate full-length cDNAs and genomic clones encoding ginS and to isolate cDNA and genomic clones of other genes that have a high identity, particularly high sequence identity, to a ginS gene. Such probes generally will comprise at least 15 nucleotide residues or base pairs. Preferably, such probes will have at least 30 nucleotide residues or base pairs and may have at least 50 nucleotide residues or base pairs. Particularly preferred probes will have at least 20 nucleotide residues or base pairs and will have lee than 30 nucleotide residues or base pairs.

A coding region of a ginS gene may be isolated by screening using a DNA sequence provided in Table 1 [SEQ ID NO:1] to synthesize an oligonucleotide probe. A labeled oligonucleotide having a sequence complementary to that of a gene of the invention is then used to screen a library of cDNA, genomic DNA or mRNA to determine which members of the library the probe hybridizes to.

There are several methods available and well known to those skilled in the art to obtain full-length DNAs, or extend short DNAs, for example those based on the method of Rapid Amplification of cDNA ends (RACE) (see, for example, Frohman, et al., Proc. Natl. Acad. Sci. USA 85: 8998-9002 (1988)). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the “missing” 5′ end of the DNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the selected gene sequence). The products of this reaction can then be analyzed by DNA sequencing and a full-length DNA constructed either by joining the product directly to the existing DNA to give a complete sequence, or carrying out a separate full-length PCR using the new sequence information for the design of the 5′ primer.

The polynucleotides and polypeptides of the invention may be employed, for example, as research reagents and materials for discovery of treatments of and diagnostics for diseases, particularly human diseases, as further discussed herein relating to polynucleotide assays.

The polynucleotides of the invention that are oligonucleotides derived from a sequence of Table 1 [SEQ ID NOS:1 or 2] may be used in the processes herein as described, but preferably for PCR, to determine whether or not the polynucleotides identified herein in whole or in part are transcribed in bacteria in infected tissue. It is recognized that such sequences will also have utility in diagnosis of the stage of infection and type of infection the pathogen has attained.

The invention also provides polynucleotides that encode a polypeptide that is a mature protein plus additional amino or carboxyl-terminal amino acids, or amino acids interior to a mature polypeptide (when a mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from a mature protein by cellular enzymes.

For each and every polynucleotide of the invention there is provided a polynucleotide complementary to it. It is preferred that these complementary polynucleotides are fully complementary to each polynucleotide with which they are complementary.

A precursor protein, having a mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

As will be recognized, the entire polypeptide encoded by an open reading frame is often not required for activity. Accordingly, it has become routine in molecular biology to map the boundaries of the primary structure required for activity with N-terminal and C-terminal deletion experiments. These experiments utilize exonuclease digestion or convenient restriction sites to cleave coding nucleic acid sequence. For example, Promega (Madison, Wis.) sell an Erase-a-base™ system that uses Exonuclease III designed to facilitate analysis of the deletion products (protocol available at www.promega.com). The digested endpoints can be repaired (e.g., by ligation to synthetic linkers) to the extent necessary to preserve an open reading frame. In this way, the nucleic acid of SEQ ID NO:1 readily provides contiguous fragments of SEQ ID NO:2 sufficient to provide an activity, such as an enzymatic, binding or antibody-inducing activity. Nucleic acid sequences encoding such fragments of SEQ ID NO:2 and variants thereof as described herein are within the invention, as are polypeptides so encoded.

In sum, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, that is a precursor to a proprotein, having a leader sequence and one or more prosequences, that generally are removed during processing steps that produce active and mature forms of the polypeptide.

Vectors, Host Cells, Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

Recombinant polypeptides of the present invention may be prepared by processes well known in those skilled in the art from genetically engineered host cells comprising expression systems. Accordingly, in a further aspect, the present invention relates to expression systems that comprise a polynucleotide or polynucleotides of the present invention, to host cells that are genetically engineered with such expression systems, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., Basic Methods In Molecular Biology (1986) and Sambrook, et al., Molecular Cloning: A Laboratory Manual, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as cells of streptococci, staphylococci, enterococci Escherichia coli, streptomyces, cyanobacteria, Bacillus subtilis, and Staphylococcus aureus; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses and retroviruses, and vectors derived from combinations thereof, such as those derived from plasmid and bacteriophage genetic elements, such as cosmids and phagemids. The expression system constructs may comprise control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., Molecular Cloning: A Laboratory Manual (1989).

In recombinant expression systems in eukaryotes, for secretion of a translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

Diagnostic, Prognostic, Serotyping and Mutation Assays

This invention is also related to the use of ginS polynucleotides and polypeptides of the invention for use as diagnostic reagents. Detection of ginS polynucleotides and/or polypeptides in a eukaryote, particularly a mammal, and especially a human, will provide a diagnostic method for diagnosis of disease, staging of disease or response of an infectious organism to drugs. Eukaryotes, particularly mammals, and especially humans, particularly those infected or suspected to be infected with an organism comprising the ginS gene or ginS protein, may be detected at the nucleic acid or amino acid level by a variety of well known techniques as well as by methods provided herein.

Polypeptides and polynucleotides for prognosis, diagnosis or other analysis may be obtained from a putatively infected and/or infected individual's bodily materials. Polynucleotides from any of these sources, particularly DNA or RNA, may be used directly for detection or may be amplified enzymatically by using PCR or any other amplification technique prior to analysis. RNA, particularly mRNA, cDNA and genomic DNA may also be used in the same ways. Using amplification, characterization of the species and strain of infectious or resident organism present in an individual, may be made by an analysis of the genotype of a selected polynucleotide of the organism. Deletions and insertions can be detected by a change in size of the amplified product in comparison to a genotype of a reference sequence selected from a related organism, preferably a different species of the same genus or a different strain of the same species. Point mutations can be identified by hybridizing amplified DNA to labeled ginS polynucleotide sequences. Perfectly or significantly matched sequences can be distinguished from imperfectly or more significantly mismatched duplexes by DNase or RNase digestion, for DNA or RNA respectively, or by detecting differences in melting temperatures or renaturation kinetics. Polynucleotide sequence differences may also be detected by alterations in the electrophoretic mobility of polynucleotide fragments in gels as compared to a reference sequence. This may be carried out with or without denaturing agents. Polynucleotide differences may also be detected by direct DNA or RNA sequencing. See, for example, Myers et al., Science, 230: 1242 (1985). Sequence changes at specific locations also may be revealed by nuclease protection assays, such as RNase, VI and SI protection assay or a chemical cleavage method. See, for example, Cotton et al., Proc. Natl. Acad. Sci., USA, 85: 4397-4401 (1985).

In another embodiment, an array of oligonucleotides probes comprising ginS nucleotide sequence or fragments thereof can be constructed to conduct efficient screening of, for example, genetic mutations, serotype, taxonomic classification or identification. Array technology methods are well known and have general applicability and can be used to address a variety of questions in molecular genetics including gene expression, genetic linkage, and genetic variability (see, for example, Chee et al., Science 274: 610 (1996).

Thus in another aspect, the present invention relates to a diagnostic kit that comprises: (a) a polynucleotide of the present invention, preferably the nucleotide sequence of SEQ ID NO:1, or a fragment thereof; (b) a nucleotide sequence complementary to that of (a); (c) a polypeptide of the present invention, preferably the polypeptide of SEQ ID NO:2 or a fragment thereof; or (d) an antibody to a polypeptide of the present invention, preferably to the polypeptide of SEQ ID NO:2. It will be appreciated that in any such kit, (a), (b), (c) or (d) may comprise a substantial component. Such a kit will be of use in diagnosing a disease or susceptibility to a Disease, among others.

This invention also relates to the use of polynucleotides of the present invention as diagnostic reagents. Detection of a mutated form of a polynucleotide of the invention, preferable, SEQ ID NO:1, that is associated with a disease or pathogenicity will provide a diagnostic tool that can add to, or define, a diagnosis of a disease, a prognosis of a course of disease, a determination of a stage of disease, or a susceptibility to a disease, that results from under-expression, over-expression or altered expression of the polynucleotide. Organisms, particularly infectious organisms, carrying mutations in such polynucleotide may be detected at the polynucleotide level by a variety of techniques, such as those described elsewhere herein.

The differences in a polynucleotide and/or polypeptide sequence between organisms possessing a first phenotype and organisms possessing a different, second different phenotype can also be determined. If a mutation is observed in some or all organisms possessing the first phenotype but not in any organisms possessing the second phenotype, then the mutation is likely to be the causative agent of the first phenotype.

Cells from an organism carrying mutations or polymorphisms (allelic variations) in a polynucleotide and/or polypeptide of the invention may also be detected at the polynucleotide or polypeptide level by a variety of techniques, to allow for serotyping, for example. For example, RT-PCR can be used to detect mutations in the RNA. It is particularly preferred to use RT-PCR in conjunction with automated detection systems, such as, for example, GeneScan. RNA, cDNA or genomic DNA may also be used for the same purpose, PCR. As an example, PCR primers complementary to a polynucleotide encoding ginS polypeptide can be used to identify and analyze mutations. The invention further provides these primers with 1, 2, 3 or 4 nucleotides removed from the 5′ and/or the 3′ end. These primers may be used for, among other things, amplifying ginS DNA and/or RNA isolated from a sample derived from an individual, such as a bodily material. The primers may be used to amplify a polynucleotide isolated from an infected individual, such that the polynucleotide may then be subject to various techniques for elucidation of the polynucleotide sequence. In this way, mutations in the polynucleotide sequence may be detected and used to diagnose and/or prognose the infection or its stage or course, or to serotype and/or classify the infectious agent.

The invention further provides a process for diagnosing, disease, preferably bacterial infections, more preferably infections caused by Porphyromonas gingivalis, comprising determining from a sample derived from an individual, such as a bodily material, an increased level of expression of polynucleotide having a sequence of Table 1 [SEQ ID NO:1]. Increased or decreased expression of a ginS polynucleotide can be measured using any on of the methods well known in the art for the quantitation of polynucleotides, such as, for example, amplification, PCR, RT-PCR, RNase protection, Northern blotting, spectrometry and other hybridization methods.

In addition, a diagnostic assay in accordance with the invention for detecting over-expression of ginS polypeptide compared to normal control tissue samples may be used to detect the presence of an infection, for example. Assay techniques that can be used to determine levels of a ginS polypeptide, in a sample derived from a host, such as a bodily material, are well-known to those of skill in the art. Such assay methods include radioimmunoassays, competitive-binding assays, Western Blot analysis, antibody sandwich assays, antibody detection and ELISA assays.

Antagonists and Agonists—Assays and Molecules

Polypeptides and polynucleotides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

Polypeptides and polynucleotides of the present invention are responsible for many biological functions, including many disease states, in particular the Diseases herein mentioned. It is therefore desirable to devise screening methods to identify compounds that agonize (e.g., stimulate) or that antagonize (e.g.,inhibit) the function of the polypeptide or polynucleotide. Accordingly, in a further aspect, the present invention provides for a method of screening compounds to identify those that agonize or that antagonize the function of a polypeptide or polynucleotide of the invention, as well as related polypeptides and polynucleotides. In general, agonists or antagonists (e.g., inhibitors) may be employed for therapeutic and prophylactic purposes for such Diseases as herein mentioned. Compounds may be identified from a variety of sources, for example, cells, cell-free preparations, chemical libraries, and natural product mixtures. Such agonists and antagonists so-identified may be natural or modified substrates, ligands, receptors, enzymes, etc, as the case may be, of ginS polypeptides and polynucleotides; or may be structural or functional mimetics thereof (see Coligan et al., Current Protocols in Immunology 1(2): Chapter 5 (1991)).

The screening methods may simply measure the binding of a candidate compound to the polypeptide or polynucleotide, or to cells or membranes bearing the polypeptide or polynucleotide, or a fusion protein of the polypeptide by means of a label directly or indirectly associated with the candidate compound. Alternatively, the screening method may involve competition with a labeled competitor. Further, these screening methods may test whether the candidate compound results in a signal generated by activation or inhibition of the polypeptide or polynucleotide, using detection systems appropriate to the cells comprising the polypeptide or polynucleotide. Inhibitors of activation are generally assayed in the presence of a known agonist and the effect on activation by the agonist by the presence of the candidate compound is observed. Constitutively active polypeptide and/or constitutively expressed polypeptides and polynucleotides may be employed in screening methods for inverse agonists, in the absence of an agonist or antagonist, by testing whether the candidate compound results in inhibition of activation of the polypeptide or polynucleotide, as the case may be. Further, the screening methods may simply comprise the steps of mixing a candidate compound with a solution comprising a polypeptide or polynucleotide of the present invention, to form a mixture, measuring ginS polypeptide and/or polynucleotide activity in the mixture, and comparing the ginS polypeptide and/or polynucleotide activity of the mixture to a standard. Fusion proteins, such as those made from Fc portion and ginS polypeptide, as herein described, can also be used for high-throughput screening assays to identify antagonists of the polypeptide of the present invention, as well as of phylogenetically and/or functionally related polypeptides (see D. Bennett et al., J Mol Recognition 8:52-58 (1995); and K. Johanson et al., J Biol Chem, 270: 9459-9471 (1995)).

The polynucleotides, polypeptides and antibodies that bind to and/or interact with a polypeptide of the present invention may also be used to configure screening methods for detecting the effect of added compounds on the production of mRNA and/or polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

The invention also provides a method of screening compounds to identify those that enhance (agonist) or block (antagonist) the action of ginS polypeptides or polynucleotides, particularly those compounds that are bacteristatic and/or bactericidal. The method of screening may involve high-throughput techniques. For example, to screen for agonists or antagonists, a synthetic reaction mix, a cellular compartment, such as a membrane, cell envelope or cell wall, or a preparation of any thereof, comprising ginS polypeptide and a labeled substrate or ligand of such polypeptide is incubated in the absence or the presence of a candidate molecule that may be a ginS agonist or antagonist. The ability of the candidate molecule to agonize or antagonize the ginS polypeptide is reflected in decreased binding of the labeled ligand or decreased production of product from such substrate. Molecules that bind gratuitously, i.e., without inducing the effects of ginS polypeptide are most likely to be good antagonists. Molecules that bind well and, as the case may be, increase the rate of product production from substrate, increase signal transduction, or increase chemical channel activity are agonists. Detection of the rate or level of, as the case may be, production of product from substrate, signal transduction, or chemical channel activity may be enhanced by using a reporter system. Reporter systems that may be useful in this regard include but are not limited to colorimetric, labeled substrate converted into product, a reporter gene that is responsive to changes in ginS polynucleotide or polypeptide activity, and binding assays known in the art.

Polypeptides of the invention may be used to identify membrane bound or soluble receptors, if any, for such polypeptide, through standard receptor binding techniques known in the art. These techniques include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (e.g., cells, cell membranes, cell supernatants, tissue extracts, bodily materials). Other methods include biophysical techniques such as surface plasmon resonance and spectroscopy. These screening methods may also be used to identify agonists and antagonists of the polypeptide that compete with the binding of the polypeptide to its receptor(s), if any. Standard methods for conducting such assays are well understood in the art.

The fluorescence polarization value for a fluorescently-tagged molecule depends on the rotational correlation time or tumbling rate. Protein complexes, such as formed by ginS polypeptide associating with another ginS polypeptide or other polypeptide, labeled to comprise a fluorescently-labeled molecule will have higher polarization values than a fluorescently labeled monomeric protein. It is preferred that this method be used to characterize small molecules that disrupt polypeptide complexes.

Fluorescence energy transfer may also be used characterize small molecules that interfere with the formation of ginS polypeptide dimers, trimers, tetramers or higher order structures, or structures formed by ginS polypeptide bound to another polypeptide. ginS polypeptide can be labeled with both a donor and acceptor fluorophore. Upon mixing of the two labeled species and excitation of the donor fluorophore, fluorescence energy transfer can be detected by observing fluorescence of the acceptor. Compounds that block dimerization will inhibit fluorescence energy transfer.

Surface plasmon resonance can be used to monitor the effect of small molecules on ginS polypeptide self-association as well as an association of ginS polypeptide and another polypeptide or small molecule. ginS polypeptide can be coupled to a sensor chip at low site density such that covalently bound molecules will be monomeric. Solution protein can then passed over the ginS polypeptide -coated surface and specific binding can be detected in real-time by monitoring the change in resonance angle caused by a change in local refractive index. This technique can be used to characterize the effect of small molecules on kinetic rates and equilibrium binding constants for ginS polypeptide self-association as well as an association of ginS polypeptide and another polypeptide or small molecule.

A scintillation proximity assay may be used to characterize the interaction between an association of ginS polypeptide with another ginS polypeptide or a different polypeptide ginS polypeptide can be coupled to a scintillation-filled bead. Addition of radio-labeled ginS polypeptide results in binding where the radioactive source molecule is in close proximity to the scintillation fluid. Thus, signal is emitted upon ginS polypeptide binding and compounds that prevent ginS polypeptide self-association or an association of ginS polypeptide and another polypeptide or small molecule will diminish signal.

In other embodiments of the invention there are provided methods for identifying compounds that bind to or otherwise interact with and inhibit or activate an activity or expression of a polypeptide and/or polynucleotide of the invention comprising: contacting a polypeptide and/or polynucleotide of the invention with a compound to be screened under conditions to permit binding to or other interaction between the compound and the polypeptide and/or polynucleotide to assess the binding to or other interaction with the compound, such binding or interaction preferably being associated with a second component capable of providing a detectable signal in response to the binding or interaction of the polypeptide and/or polynucleotide with the compound; and determining whether the compound binds to or otherwise interacts with and activates or inhibits an activity or expression of the polypeptide and/or polynucleotide by detecting the presence or absence of a signal generated from the binding or interaction of the compound with the polypeptide and/or polynucleotide.

Another example of an assay for ginS agonists is a competitive assay that combines ginS and a potential agonist with ginS-binding molecules, recombinant ginS binding molecules, natural substrates or ligands, or substrate or ligand mimetics, under appropriate conditions for a competitive inhibition assay. ginS can be labeled, such as by radioactivity or a calorimetric compound, such that the number of ginS molecules bound to a binding molecule or converted to product can be determined accurately to assess the effectiveness of the potential antagonist.

It will be readily appreciated by the skilled artisan that a polypeptide and/or polynucleotide of the present invention may also be used in a method for the structure-based design of an agonist or antagonist of the polypeptide and/or polynucleotide, by: (a) determining in the first instance the three-dimensional structure of the polypeptide and/or polynucleotide, or complexes thereof; (b) deducing the three-dimensional structure for the likely reactive site(s), binding site(s) or motif(s) of an agonist or antagonist; (c) synthesizing candidate compounds that are predicted to bind to or react with the deduced binding site(s), reactive site(s), and/or motif(s); and (d) testing whether the candidate compounds are indeed agonists or antagonists. It will be further appreciated that this will normally be an iterative process, and this iterative process may be performed using automated and computer-controlled steps.

In a further aspect, the present invention provides methods of treating abnormal conditions such as, for instance, a Disease, related to either an excess of, an under-expression of, an elevated activity of, or a decreased activity of ginS polypeptide and/or polynucleotide.

If the expression and/or activity of the polypeptide and/or polynucleotide is in excess, several approaches are available. One approach comprises administering to an individual in need thereof an inhibitor compound (antagonist) as herein described, optionally in combination with a pharmaceutically acceptable carrier, in an amount effective to inhibit the function and/or expression of the polypeptide and/or polynucleotide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide and/or polynucleotide may be administered. Typical examples of such competitors include fragments of the ginS polypeptide and/or polypeptide.

In still another approach, expression of the gene encoding endogenous ginS polypeptide can be inhibited using expression blocking techniques. This blocking may be targeted against any step in gene expression, but is preferably targeted against transcription and/or translation. An examples of a known technique of this sort involve the use of antisense sequences, either internally generated or separately administered (see, for example, O'Connor, J Neurochem 56: 560 (1991) in Oligodeoxynucleotides as Antisense Inhibitors of Gene Expression, CRC Press, Boca Raton, Fla. (1988)). Alternatively, oligonucleotides that form triple helices with the gene can be supplied (see, for example, Lee et al., Nucleic Acids Res 6:3073 (1979); Cooney et al., Science 241:456 (1988); Dervan et al., Science 251:1360 (1991)). These oligomers can be administered per se or the relevant oligomers can be expressed in vivo.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Delgamo or other translation facilitating sequences of the respective mRNA can be used to construct antisense sequences to control the expression of the coding sequence of interest.

The invention also provides the use of the polypeptide, polynucleotide, agonist or antagonist of the invention to interfere with the initial physical interaction between a pathogen or pathogens and a eukaryotic, preferably mammalian, host responsible for sequelae of infection. In particular, the molecules of the invention may be used: in the prevention of adhesion of bacteria, in particular gram positive and/or gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial ginS proteins that mediate tissue damage and/or; to block the normal progression of pathogenesis in infections initiated other than by the implantation of in-dwelling devices or by other surgical techniques.

In accordance with yet another aspect of the invention, there are provided ginS agonists and antagonists, preferably bacteristatic or bactericidal agonists and antagonists.

The antagonists and agonists of the invention may be employed, for instance, to prevent, inhibit and/or treat diseases.

Helicobacter pylori (herein “H.pylori”) bacteria infect the stomachs of over one-third of the world's population causing stomach cancer, ulcers, and gastritis (International Agency for Research on Cancer (1994) Schistosomes, Liver Flukes and Helicobacter Pylori (International Agency for Research on Cancer, Lyon, France, http://www.uicc.ch/ecp/ecp2904.htm). Moreover, the International Agency for Research on Cancer recently recognized a cause-and-effect relationship between H.pylori and gastric adenocarcinoma, classifying the bacterium as a Group I (definite) carcinogen. Preferred antimicrobial compounds of the invention (agonists and antagonists of ginS polypeptides and/or polynucleotides) found using screens provided by the invention, or known in the art, particularly narrow-spectrum antibiotics, should be useful in the treatment of H.pylori infection. Such treatment should decrease the advent of H.pylori-induced cancers, such as gastrointestinal carcinoma. Such treatment should also prevent, inhibit and/or cure gastric ulcers and gastritis.

Glossary

The following definitions are provided to facilitate understanding of certain terms used frequently herein.

“Bodily material(s) means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

“Disease(s)” means any disease caused by or related to infection by a bacteria, including, for example, disease, such as, infections of the upper respiratory tract (e.g, otitis media, bacterial tracheitis, acute epiglottitis, thyroiditis), lower respiratory (e.g, empyema, lung abscess), cardiac (e.g, infective endocarditis), gastrointestinal (e.g., secretory diarrhoea, splenic absces, retroperitoneal abscess), CNS (e.g., cerebral abscess), eye (e.g., blepharitis, conjunctivitis, keratitis, endophthalmitis, preseptal and orbital cellulitis, darcryocystitis), kidney and urinary tract (e.g, epididymitis, intrarenal and perinephric absces, toxic shock syndrome), skin (e.g., impetigo, folliculitis, cutaneous abscesses, cellulitis, wound infection, bacterial myositis) bone and joint (e.g, septic arthritis, osteomyelitis).

“Host cell(s)” is a cell that has been introduced (e.g, transformed or transfected) or is capable of introduction (e.g, transformation or transfection) by an exogenous polynucleotide sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12: 387 (1984)), BLASTP, BLASTN, and FASTA (Altschul, S. F. et al., J. Molec. Biol. 215: 403-410 (1990)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990)). The well known Smith Waterman algorithm may also be used to determine identity.

Parameters for polypeptide sequence comparison include the following: Algorithm: Needleman and Wunsch, J Mol Biol. 48: 443-453 (1970)). Comparison matrix: BLOSSUM62 from Hentikoff and Hentikoff, Proc. Natl. Acad. Sci. USA. 89:10915-10919 (1992)).

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch, J. Mol Biol. 48: 443-453 (1970).

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 95, 97 or 100% identity to the reference sequence of SEQ ID NO:1, wherein said polynucleotide sequence may be identical to the reference sequence of SEQ ID NO:1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO:1 by the integer defining the percent identity divided by 100 and then subtracting that product from said total number of nucleotides in SEQ ID NO:1, or:

nₙ≦xₙ−(xₙ·y),

wherein nₙ is the number of nucleotide alterations, xₙ is the total number of nucleotides in SEQ ID NO:1, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:

nₐ≦xₐ—(xₐ·y),

wherein nₐ is the number of amino acid alterations, xₐ is the total number of amino acids in SEQ ID NO:2, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Organism(s)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterni, Shigella flexneri, Pseudomonas aerutginosa, Franscisella tularensis, Bricella abortis, Bacillus anthracis, Bacillus cereus, Clostridium perfringens, Clostridium tetani, Clostridium botulinum, Treponema pallidum, Rickettsia rickettsii, Porphyromonas gingivalis and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins: Structure and Molecular Properties, 2nd Ed., T. E. Creighton, W. H. Freeman and Company, New York (1993) and Wold, F., Posttranslational Protein Modifications: Perspectives and Prospects, pgs. 1-12 in Post-translational Covalent Modification of Proteins, B. C. Johnson, Ed., Academic Press, New York (1983); Seifter et al., Meth. Enzymol. 182: 626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced or transformed into a host cell or host cell lysate for the production of the polynucleotides and polypeptides of the invention.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled altisans.

### EXAMPLES

The examples below are carried out using standard techniques, that are well known and routine to those of skill in the art, except where otherwise described in detail. The examples are illustrative, but do not limit the invention.

### Example 1

### Bacterial Strain Selection, Plasmid Curation, Library Production and DNA Sequencing

Porphyromonas gingivalis W50 was maintained on Fastidious Anaerobic Agar (LabM) plates containing 5% (v/v) horse blood (TCS, Buckingham, U.K.) at 37° C. in an anaerobic cabinet (MK3 Anaerobic Work Station, Dow Scientific) equilibrated in 80% nitrogen/10% hydrogen/10% carbon dioxide. Liquid cultures of Porphyromonas gingivalis W50 were grown anaerobically at 37° C. in Brain Heart Infusion (BHI) broth (Oxoid) containing 5 μg/ml (w/v) haemin. The wildtype and mutant strains of Porphyromonas gingivalis W50 were maintained as frozen bead stocks (PRO-LAB Diagnostics) and stored at −80° C. Freshly isolated colonies were used for each experiment and incubation on plates in the anaerobic cabinet was allowed to proceed for 4-5 days before inoculating into broth. The Porphyromonas gingivalis W50 genomic library was constructed in pBluescript SK⁺ (Stratagene).

Other bacterial strains used in this study are listed in Table 2.

### TABLE 2.

### Bacterial Strains and Plasmid Reporter Constructs used for Autoinducer Detection

TABLE 2
Bacterial Strains and Plasmid Reporter Constructs used for
Autoinducer Detection.

| Plasmid/ | | |
|---|---|---|
| Strain | Description | Source/Ref. |
| P. gingivalis | Wildtype strain | Shah et al, 1989 |
| W50 | | |
| V. harveyi | Wildtype strain | Bassler et al, |
| BB120 | | 1997 |
| V. harveyi | Mutant strain which responds to AI-2 | Greenberg et al, |
| BB170 | | 1979 |
| V. harveyi | Mutant strain which responds to AI-1 | Greenberg et al, |
| BB886 | | 1979 |
| CV026 | Double Tn5 mutant derived from | Winson et al, |
| | C. violaeum ATCC 31532; N-acyl-HSL | 1994 |
| | sensor, Km^{R}, Hg^{R} | |
| pSB401 | N-acyl HSL plasmid containing a | Winson et al, |
| | luxRI′::CDA fusion in pACYC 184, | 1998a, b |
| | Tet^{R} | |
| pSB536 | N-acyl HSL plasmid containing an | Swift et al, 1997 |
| | ahyRI′::luxCDABE fusion in pRK415 | |
| | Amp^{R} | |
| pSB1142 | N-acyl HSL plasmid containing a | |
| | lasRI′::luxCDABE fusion in pRK415, | |
| | Tet^{R} | |
| pSB1075 | N-acyl HSL plasmid containing a | Winson et al, |
| | lasRI′::luxCDABE fusion in pUC18, | 1998a, b |
| | Tet^{R} | |

All of the plasmid reporters introduced into E.coli JM109 were grown at 37° C. in Luria-Bertani medium (LB), containing 10 g bacto-tryptone (Difco), 5 g yeast extract (Difco) and 5 g NaCl (Sigma) per litre. The V.harveyi strains were grown at 30° C. in AB medium, the recipe for which has been previously reported (Greenberg et al, 1979). Antibiotics were used at the following concentrations (mg/litre); ampicillin (Amp), 50; clindamycin (Cn), 5; erythromycin (Erm), 300; tetracycline (Tet), 10. DNA isolation, restriction digests and transformation of E.coli was performed as described by Sambrook et al (1989). Bacterial chromosomal DNA was purified using the CTAB method. Probes for Southern blot analysis were labelled by using the DIG-labelling system of Boerhinger Mannheim.

The polynucleotide having a DNA sequence given in Table 1 [SEQ ID NO: 1] was obtained from a library of clones of chromosomal DNA of Porphyromonas gingivalis in E.coli. The sequencing data from two or more clones comprising overlapping Porphyromnonas gingivalis DNAs was used to construct the contiguous DNA sequence in SEQ ID NO:1. Libraries may be prepared by routine methods, for example:

Methods 1 and 2 below

Total cellular DNA is isolated from Porphyromonas gingivalis W50 according to standard procedures and size-fractionated by either of two methods.

Method 1

Total cellular DNA is mechanically sheared by passage through a needle in order to size-fractionate according to standard procedures. DNA fragments of up to 11 kbp in size are rendered blunt by treatment with exonuclease and DNA polymerase, and EcoRI linkers added. Fragments are ligated into the vector Lambda ZapII that has been cut with EcoRI, the library packaged by standard procedures and E.coli infected with the packaged library. The library is amplified by standard procedures.

Method 2

Total cellular DNA is partially hydrolyzed with a one or a combination of restriction enzymes appropriate to generate a series of fragments for cloning into library vectors (e.g, RsaI, PaII, AluI, Bsh1235I), and such fragments are size-fractionated according to standard procedures. EcoRI linkers are ligated to the DNA and the fragments then ligated into the vector Lambda ZapII that have been cut with EcoRI, the library packaged by standard procedures, and Escherichia coli infected with the packaged library. The library is amplified by standard procedures.

### Example 2

### N-acyl Homoserine Lactone Assays

Chromobacterium (CV026) forward and reverse assays and thin-layer chromatography of concentrated culture supernatants using CV026 and bioluminescent reporters have been previously reported (McClean et al, 1997; Swift et al, 1997; Winson et al, 1998). Screening for detection of luminescence was carried out using a Luminograph LB980 photon imaging camera (EG&G Berthold) according to the Manufacturer's instructions.

### Example 3

### Molecular Cloning of ginS Polynucleotide

PCR amplification of ginS from P.gingivalis W50 chromosomal DNA was carried out using primers Por11 5′-GTATTATCAGCGGAATTCCCGGCGAAGGTCG-3′ [SEQ ID NO:3] and Por12 5′-GATACCGCCTCCGGATCCAATAATCCATCCGG-3′ [SEQ ID NO:4] which were designed to the P.gingivalis W83 genome sequence and contained created EcoRI and BamHI restriction sites respectively. The PCR product was purified using a Qiagen PCR purification kit according to the manufacturer's instructions, ligated into pHG327, previously digested with EcoRI and BamHII, (Stewart et al, 1986) and electroporated into E.coli DH5α (Sambrook et al, 1989), thus creating pHGin1.

### Example 4

### Mutagenesis of ginS in E.coli DH5α

A deletion mutant of ginS was prepared by chimaeric PCR to remove 77 amino-acids. Two PCR products were generated from chromosomal DNA, containing NotI restriction sites, one with primers Por11 and Por13B 5′-GCGGCCGCCACCAAATGCTCGATCGTATGCCAG-3′ [SEQ ID NO:5] the other with primers Por14B 5′-TGGCGGCCGCGCGTGAGGTACTCGATGTAGG-3′ [SEQ ID NO:6] and Por12. Subsequently, 1 μl of each of these purified PCR products served as a template in a second PCR amplification using primers Por11 and Por12. The PCR product was digested with BamHI and EcoRI, purified, ligated into pHG327 (similarly digested) and electroporated into E.coli DH5α, to create pHGin2.

### Example 5

### Autoinducer-2 Assay

The AI-2 bioassay using the V.harveyi biosensor BB170 (sensor 1⁻, sensor 2⁺) has been reported previously (Greenberg et al, Arch. Micro. 120: 87-91 (1979).

P.gingivalis W50 was grown in a chemically defined medium (Milner et al, FEMS Microbiol. Letts. 140: 125-130 (1996), E.coli DH5α in LB medium and the V.harveyi strains in AB medium and cell-free culture supernatants were added to V.harveyi BB170 suspension in AB medium at 10% (v/v). AI-2 activity is reported as Relative Light Units (RLU), measured in an EG&G Wallac Victor luminometer. Following incubation for the time indicated.

### Example 6

### Expression of ginS

PCR amplification of ginS from P.gingivalis W50 chromosomal DNA was also carried out using primers Por19a 5′-AGACAATCCCGAATTCGAGATGGAA-3′ [SEQ ID NO:7] and Por20a 5′-TGAGAAATAGAGCGGATCCTAAGC-3′ [SEQ ID NO:8] which contained EcoRI and BamHI restriction sites respectively. The product was purified, ligated into pMal-c2 (New England BioLabs) using the EcoRI/BamHI sites and electroporated into E.coli DH50α, thus creating pMalGin1. DH5α (pMalGin1) was grown to an A600 of 0.2, 0.3 mM IPTG was added and growth was continued until an A600 of 1.2. The cells were harvested by centrifugation at 10,000×g for 10 min, resuspended in PBS and sonicated. Insoluble material was removed by centrifugation at 4,000×g for 5 min. Purification was carried out by affinity chromatography using an amylose resin column (New England BioLabs) and elution in 10 mM maltose according to the manufacturer's instruction.

### Example 7

### TCA Precipitation and SDS-PAGE Analysis

Cultures of P.gingivalis wildtype (strain W50) and the ginS mutant strain were TCA precipitated by adding 10% (v/v) of 100% TCA (w/v) in H₂O and placing on ice for 1 h. Precipitated proteins were harvested by centrifugation at 10,000×g for 15 min and washed with ice cold acetone. Pellets were dried in air and resuspended in PBS. Sodium dodecyl sulphate (SDS) sample buffer was added and the proteins were separated by SDS-Polyacrylamide Gel Electrophoresis (PAGE) on 12.5% gels.

### Example 8

### Purification of GinS for Antibody Production

Factor Xₐ enzyme (New England BioLabs) was diluted to 200 μg/ml in PBS and 800 μg of purified MalE-ginS at a concentration of 1.2 mg/ml was incubated with the enzyme at room temperature for either 2, 4, 6 or 22 h, after which time, SDS sample buffer was added and the samples were analysed by SDS-PAGE. Purified and cleaved ginS was excised from an SDS-PAGE gel and electroeluted according to the manufacturer's instructions (BioRad). Polyclonal and monoclonal antibodies were raised against ginS in rabbits and mice respectively (Institute of Infections and Immunity, Queens' Medical Centre, Nottingham, UK). Rabbits were subcutaneously injected with 10-50 μg of purified protein four times, at two weekly intervals. Serum from the rabbits was left overnight at 4° C. to clot and then centrifuged at 3,000×g for 15 min to remove remaining red blood cells (Goding, 1980). To adsorb non-specific antibodies from polyclonal ginS serum, 200 ml of E.coli DH5α (pMalc2) was grown in LB and centrifuged at 10,000×g to harvest the cells. The cells were resuspended in 20 ml PBS and lysed in a French press. The lysate (2 ml) was mixed with 2 ml of rabbit anti-ginS serum at room temperature overnight. Azide (0.02%) was added to prevent contamination.

### Example 9

### Western Blot Analysis

Western blots were carried out, as described by Hardie, K. R. et al, 1996, except phosphate buffered saline (PBS) containing 0.5% Tween-20 was used instead of TBST, on cell lysates throughout the growth curve of E.coli DH5α, P.gingivalis wildtype and ginS mutant. The protease inhibitor, N-p-tosyl-L-lysine chloromethyl ketone (TLCK) was added at a concentration of 50 mM to cells of P.gingivalis prior to cell lysis. Samples were boiled, resolved by SDS-PAGE and transferred to nitrocelluose. The primary antibody, ginS polyclonal serum was used at 1:10,000 followed by rabbit Protein-A-alkaline phosphatase secondary antibody at 1:1000 and the blots were developed using either Amersham ECL kit or SigmaFast tablets according to the manufacturer's instructions.

### Example 10

### Generation of a P.gingivalis ginS⁻ Null Mutant

Plasmid pHGin2 was digested with EcoRI and BamHI to release the shortened version of ginS. The fragment was purifed from an agarose gel and subcloned into similarly digested pUC18 (Pharmacia), to create the plasmid pHGin18. Plasmid pVA2198 was digested with EcoRI and BamnHI to release an erythromycin cassette (erm), which was subsequently purified and ligated into pUC18Not (Herrero et al, 1990) at similar restriction sites. The erm cassette was isolated from pUC18Not by digestion with NotI and subsequently cloned into the NotI site of pHGin18, thus creating pGinerm. This construct was electroporated into P.gingivalis W50 as described by Rangarajan et al (Rangarajan, et al, Mol. Micro. 23: 955-965 (1997). Transformants were selected on FAA containing clindamycin.

### Example 11

### N-Terminal Sequence Analysis

Cell-free culture supernatant (30 ml) from the P.gingivalis W50 wildtype strain was TCA precipitated and resuspended in 400 μl of PBS. SDS sample buffer was added and 6×15 μl aliquots of protein were resolved on a 12.5% SDS-polyacrylamide gel. The proteins were blotted onto Hybond-P PVDF membrane (Amersham Life Science) in CAPS buffer using an XCell II unit supplied by Novex and run at 100 mA overnight. The membrane was stained in Naphthalene black briefly, then destained in distilled water. Proteins of approximately 55 and 48 kDa were excised from the membrane and protein sequencing was carried out using an ABI 473A Protein Sequencer according to the manufacturer's instructions (Perkin Elmer Corp., Foster City, La., USA) by the Nottingham Automated Sequencing Facility.

### Example 12

### Protease Assay

Protease activity was measured according to the method described by Rangarajan et al (Rangarajan et al, Mol. Micro. 23: 955-965 (1997). Using an ELISA plate reader (Labsystems iEMS Reader MF) at 30° C., the reaction containing the enzyme sample in 1 ml of 0.5 mM DL-BApNA/10 mM L-cysteine/10 mM CaCl₂/100 mM Tris/HCl buffer (pH 8.1) was monitored by increase in absorbance at 405 nm due to p-nitroanilide release.

### Example 13

### Haemagglutination Assay

Sheep erythrocytes (SRBC) were harvested and washed twice in PBS at 4° C. by centrifugation at 2,000×g for 3 min and diluted to 0.5% (v/v) in PBS. P.gingivalis W50 wildtype and ginS mutant were grown to an A₅₈₀ of between 0.6 and 1.0, washed once in PBS and resuspended to an A₅₈₀ of 1.0. Bacterial cells (50 μl) were added to the wells of V-shaped, 96-well plates and doubling dilutions were prepared across the plates. The SRBC (50 μl) were added and the plates were covered and incubated at 4° C. overnight. Lack of haemagglutination of SRBC was observed as a dark red pellet in the centre of the well. E.coli DH5α cells were used as a non-agglutination control and the P.gingivalis wildtype was a positive indicator of agglutination. The plates were examined and scored visually.

### Example 14

### P.gingivalis Does Not Possess A Gene(s) Homologous to V.fischeri LuxR/I

Many Gram-negative bacteria use homologues of the Vibrio fischeri quorum sensing luxR/I genes (Swift et al, TIBS 21: 214-219 (1996). The LuxI proteins synthesize AHL molecules which can be detected in spent culture supernatants using a variety of biosensors. These biosensors respond to a particular range of AHLs depending on the length of their acyl side chains. Spent culture supernatants from P.gingivalis W50 were assayed for production of short and long chain AHLs using the Chromobacterium mutant, CV026, and E.coli containing the luxCDABE-based plasmid reporters, pSB401, pSB1075 and pSB1142. This was carried out using several methods including plate assays and by overlaying Thin-Layer Chromatography plates to separate AHLs and prevent inhibition of one by the other. No production of violacin pigment or induction of luminescence, using a Luminograph LB980 photon imaging camera, was detected. The unfinished P.gingivalis W83 genome sequence database (www.forsyth.org/-pggp/) was searched for homologues of LuxR/I using the NCBI Blast server and the TBLASTN program but no homologues have been found as yet. In parallel, the existence of functional homologues was investigated by screening a genomic library against the full range of AHL biosensors. This approach also failed to detect any AHL production.

### Example 15

### Identification of a luxS Homologue in P.gingivalis

In 1999, Surette et al reported that a gene, luxS, is responsible for production of a signalling molecule (AI-2) by Vibrio harveyi. Homologues of luxS are also found in many other bacteria including Escherichia coli and Salmonella typhimurium (Surette et al, 1999a & b). A TBLASTN search of the P.gingivalis genome database with V.harveyi LuxS revealed a luxS homologue, which codes for a protein of 159 amino acids and has since been designated, ginS. The protein sequence of GinS (predicted to be 18.5 kDa) bears low amino-acid homology (30%) with other LuxS proteins from E.coli strains and Helicobacter pylori (Surette et al, 1999a). GinS is most closely related (50% identity) to the LuxS of Borrelia burgdorferi, which causes Lyme disease (see Example 16).

### Example 16

### Amino-Acid Homology Between GinS and Borrelia burgorferi LuxS from BLAST Search

Locus: LUXS_BORBU 157 aa MAY 30, 2000

Definition: Autoinducer-2 production protein, luxS (AI-2 synthesis protein).

Accession No. 050164

PID: g7387852 Version 050164 gi:7387852

Database Source: SwissProt

Source: Lyme disease spirochete, Borrelia burgorferi.

>sp|O501641|LUXS BORBU Autoinducer-2 Production Protein LuxS (AI-2 Synthesis Protein)

Length=157 aa

Score=165 bits (413), Expect=3e-40

Identities=79/158 (50%), Positives=108/158 (68%), Gaps=1/158 (0%)

Query: 1 (ginS)[SEQ ID NO:9]

| | |
|---|---|
| MEKIPSFQLDHIRLKRGIYVSRKDYIGGEVVTTFDIRMKEPNREPVLGAPELHTIEHLAA | 60 |
| M+KI SF +DH+L GIYVSRKD +TT DIR+K PN EP++ +HTIEH A | |
| MKKITSFTIDHTKLNPGIYVSRKDTFENVIFTTIDIRIKAPNIEPIIENAAIHTIEHIGA 60 | |
| Sbjct: 1 (luxS) | |

Query: 61 (ginS)[SEQ ID NO:10]

| | |
|---|---|
| TYLRNHPLYKDRIVFWGPMGCLTGNYFLMRGDYVSKDILPLMQETFRFIRDFEGEVPGTE | 120 |
| T LRN+++++IV++GPMGC TG Y ++GDY SKD++L+ F I+F+PG | |
| TLLRNNEVWTEKIVYFGPMGCRTGFYLIIFGDYESKDLVDLVSWLFSEIVNFSEPIPGAS | 120 |
| Sbjct: 61 (luxS) | |

Query: 121 (ginS)[SEQ ID NO:11]

| | |
|---|---|
| PRDCGNCLLHNLPMAKYEAEKYLREVLDVATEENLNYP | 158 |
| ++CGN HNL MAKYE+KYL ++L+EENL YP | |
| DKECGNYKEHNLDMAKYESSKYL−QILNNIKENELKYP | 157 |
| Sbjct: 121 (luxS) | |

Sbjct: 121 (luxS)

### Example 17

### Complementation of AI-2 Production in E.coli DH5α

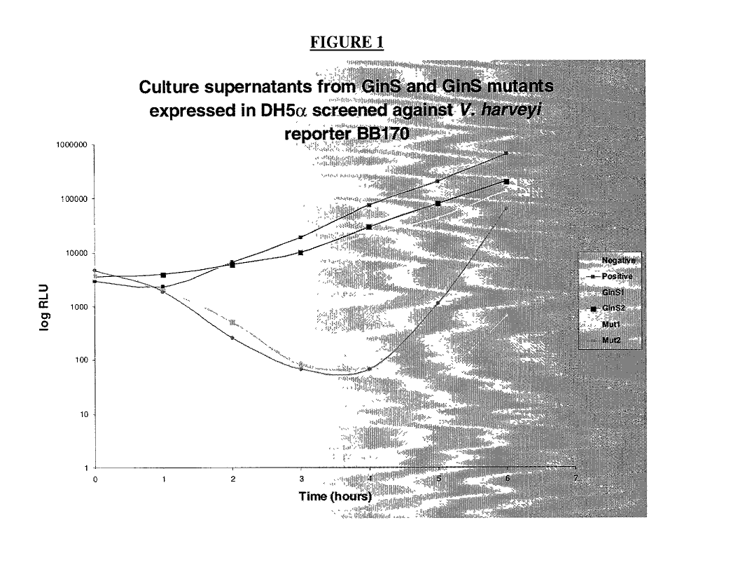

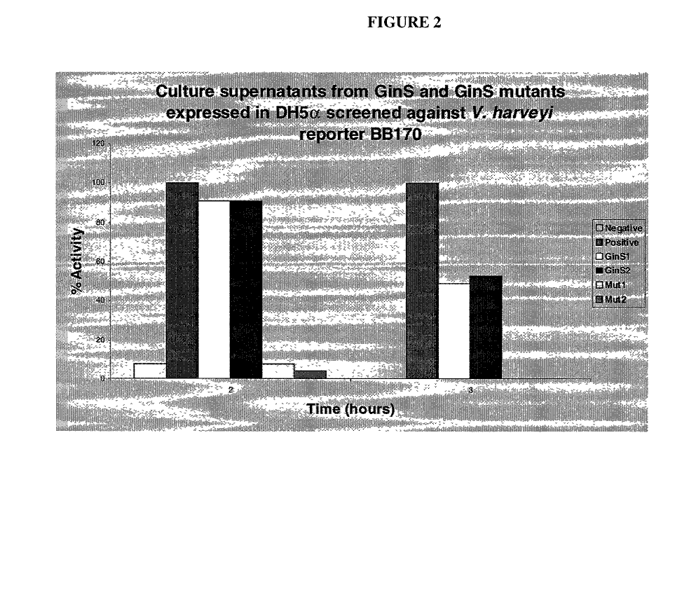

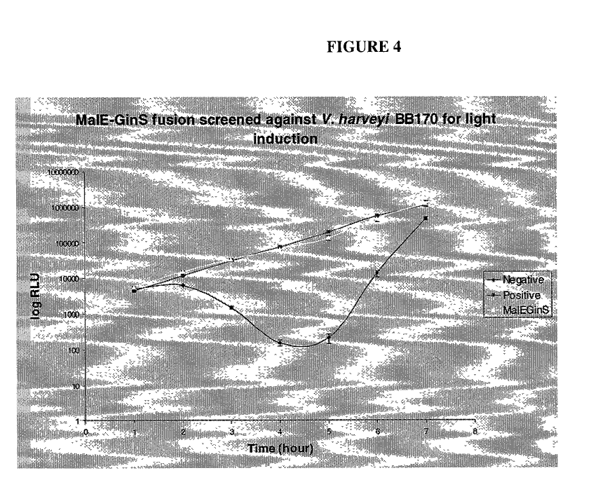

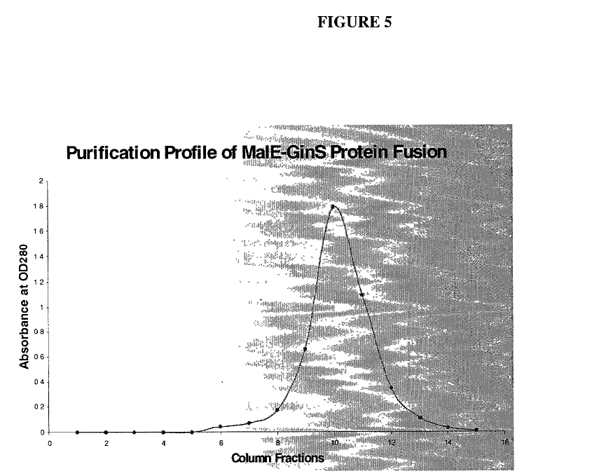

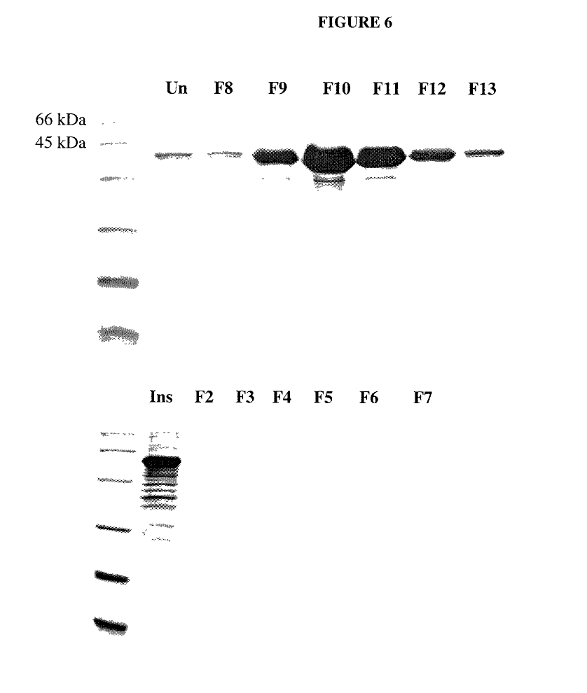

We tested whether ginS had sufficient homology to LuXSEC to complement the mutation in DH50α. GinS was amplified from P.gingivalis W50 genomic DNA and cloned into pHG327 to create pGin1. SDS-PAGE analysis of whole cell extracts of E.coli DH5α (pHGin1) failed to reveal the production of a protein at the predicted MW for GinS (18.5 kDa). However, production of AI-2 was detected in spent culture supernatant using the V.harveyi BB170 (see FIGS. 1 and 2). Consequently, ginS was cloned into pMal-c2 (pMalGin 1) and MalE-GinS (ca. 62 kDa) was produced in DH5α Analysis of spent culture supernatant of this strain using the same V.harveyi BB170 sensor, likewise demonstrated functional complementation of the LuxS_{Ec} mutation in DH5α by production of the AI-2 molecule (see FIG. 4). The functionally active MalE-GinS was subsequently purified by affinity chromatography (FIG. 5). Cleavage of GinS from MalE was successfully performed using Factor Xa as described in materials and methods (see FIG. 6). This purified GinS was used to raise specific polyclonal antisera to GinS in rabbits. Western blotting of whole cell extracts of E.coli DH5α (pMalGin1) and analysis of spent culture supernatant from E.coli DH5α (pMalGin1) supernatants at time-points throughout the growth curve, showed that AI-2 production commences within 4 of growth (mid-exponential phase) and continues through stationary phase, with some degradation evident by 9 hours (see FIG. 8).

### Confirmation of GinS Expression and AI-2 Production in E.coli and P.gingivalis.

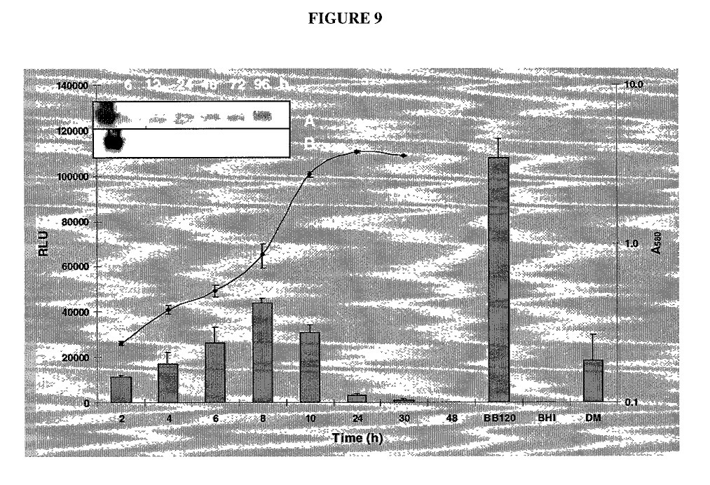

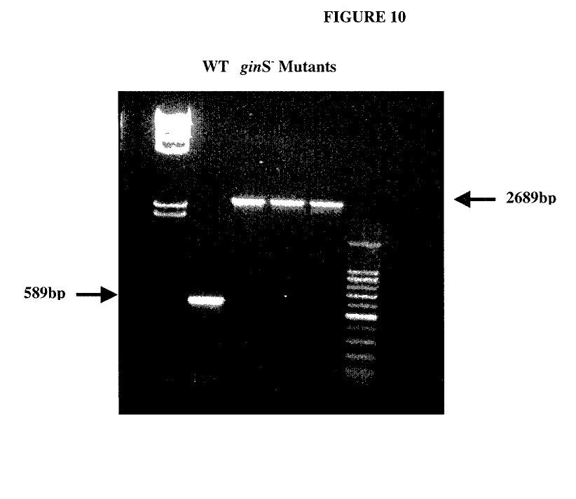

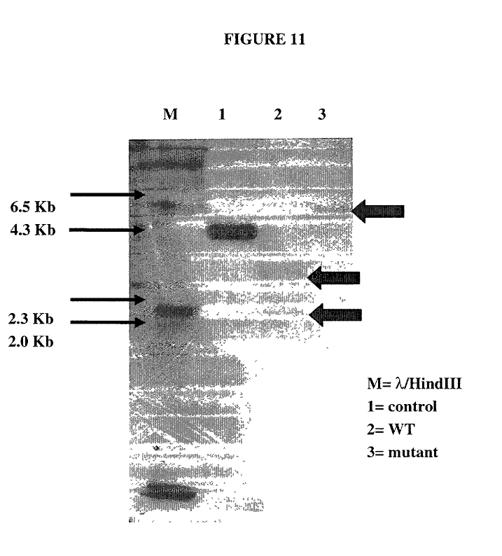

P.gingivalis W50 wildtype spent BHI culture supernatant, taken at various points throughout the growth curve, was assayed for production of AI-2 against V.harveyi BB170, but no activity was detected. However, when grown in a defined medium, the molecule was maximally detected at mid-expotential phase of growth whilst Western blots revealed that in E.coli, GinS accumulates throughout growth and is still increasing at 24 hours (see FIG. 8). In P.gingivalis, GinS is detected first at 6 hours and appears to still be increasing at 96 hours (see FIG. 9). A ginS⁻ null mutant of P.gingivalis was prepared as described in materials and methods and confirmed by PCR by the presence of a larger amplified DNA fragment corresponding to insertion of the erm cassette (FIG. 10). Southern blot analysis confirmed a single erm insertion in the chromosome (FIG. 11). Genomic DNA was digested by BalI which cuts once within ginS. This site is removed by the mutation. The digested DNA was probed with a DIG-labelled ginS PCR fragment. Wildtype genomic DNA showed two fragments hybridised to the probe as expected, whereas with the mutant DNA, a single, larger fragment was detected showing that the erm cassette had replaced the BalI restriction site. Amplification and cloning of the 5′ and 3′ regions of the ginS⁻ mutant, followed by sequencing of the clones, further confirmed that the ginS mutant had inserted into the correct region of the chromosome. The absence of GinS in Western blots of the ginS⁻ mutant further confirmed deletion of the gene (FIG. 9, Panel B).

### GinS Regulates Expression and Secretion of Rgp Proteases (Gingipains), as well as

### Haemagglutination of Erythrocytes in P.gingivalis

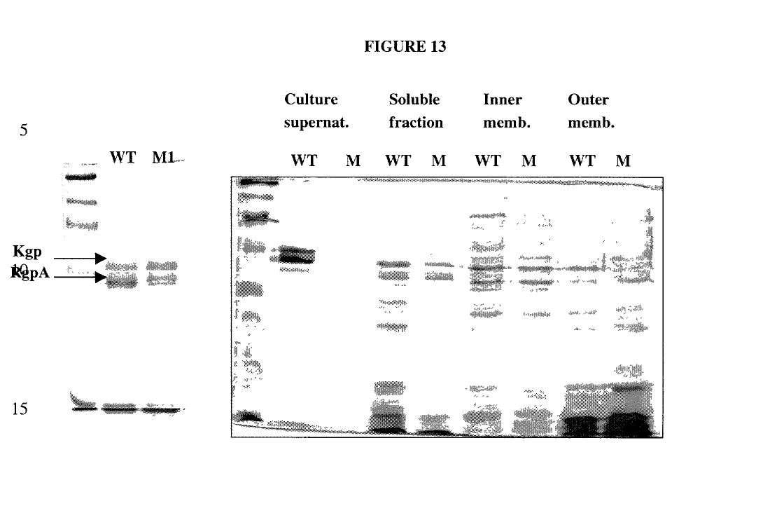

The major virulence factors produced by P.gingivalis are the secreted proteases or gingipains, Rgp and Kgp. To discover whether GinS is involved in protease regulation, the protease profile and activity of the wildtype and ginS⁻ null mutant of P.gingivalis was compared by SDS-PAGE and protease assays using BApNA and Z-Lys-pNA. SDS-PAGE analysis of TCA precipitated spent culture supernatants of the wildtype and ginS⁻ mutant revealed a down-regulation of two proteins (55 and 48 kDa) in the ginS⁻ null mutant (see FIG. 13).

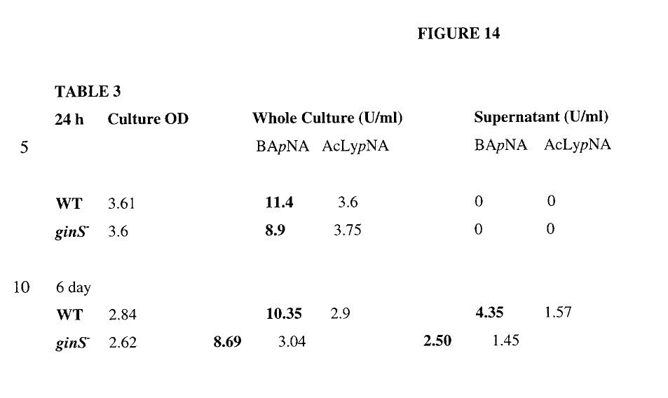

N-terminal sequencing of these proteins from the wildtype revealed sequences of DVYTDHGDLYNT [SEQ ID NO:12] and YTPVEEKQNGRM [SEQ ID NO:13] respectively, identifying the proteins as Rgp and Kgp. BApNA and Z-Lys-pNA specific assays of total cultures, cell-free supernatants and lysates revealed a significant decrease in protease activity of the ginS⁻ mutant when compared with the wildtype (see FIG. 14)

Initial analysis of KGP and RGP activity in the soluble cellular fractions demonstrated an accumulation in the mutant vs. the wildtype, suggesting that overall expression of proteases are unaffected by the insertional mutation in ginS⁻, but rather these data strongly suggest a role for GinS the regulation of protease export/secretion from the cell. However, the more detailed data obtained via analysis of the enzymatic activity in whole cell lysates (see FIG. 14 (Table 3)) analysis clearly indicates that in addition to a possible influence on protease secretion/export, RgpB expression itself and possibly also RgpA, are down-regulated in the ginS⁻ null mutant, suggesting that ginS⁻ is directly involved in regulating gene expression of an important virulence determinants directly involved in host tissue destruction. 2-D gel electrophoresis was carried out according to a standard protocol (see Berkelman, et al., 2-D Electrophoresis: Principles and Methods. Amersham Pharmacia Biotech (ftp://ftp.hpb.com/pdf/2D_Brochure_{—}13.pdf), in order to identify differences in the protein expression patterns between wildtype P.gingivalis and the ginS⁻ null mutant which might indicate regulatory control by AI-2, produced as a result of ginS expression. A single spot, designated WT2, was identified as being expressed exclusively in the wildtype and was excised and subjected to further analysis and characterization in order to obtain an identification (see Examples 30 and 31).

Methods: Sample Preparation for Mass Spectrometry

In-gel digestion of the proteins was performed on a automated digester (DigestPro™, AbiMed, Germany) using a standard protocol. After extraction of peptides from the gel, the solution was dried in a vacuum centrifuge. The peptides were resuspended in 10 μl 5% formic acid. For Nanospray MS analysis, five microliter of this solution were purified using a 10 μl pipette tip loaded with a C18 resin (ZipTip, Millipore). The bed volume of the resin of 0.6 μl was reduced by approximately half by cutting of a small piece form the front of the tip. The pipette tip was wetted with water/acetonitrile (1:2) and equilibrated with aqueous 0.2% trifluoroacetic acid. The peptides were loaded by repeatedly aspirating and dispensing the 5 μl peptide solution. The tip was washed by aspirating and dispensing of 5% formic acid for four to five times. Peptides were eluted in 1-2 μl of water/methanol (1:1) containing 5% formic acid. The eluate was immediately loaded into a nanospray microcapillary (type “N”, Protana, Odense, Denmark). After opening of the tip by touching a glass slide under a stereomicroscope, the microcapillary was placed in the ion source of a Qtof mass spectrometer for analysis.

Mass spectrometry. MALDI Tof MS was performed on a TofSpec SE instrument from Micromass, Manchester, UK. Nanospray MS and MS/MS experiments were performed on a orthogonal acceleration quadrupole-time-of-flight mass spectrometer (Q-Tof, Micromass, Manchester, UK) equipped with a Z-spray ion source for Nanospray analysis.

Database Searches

Peptide mass searches and sequence tag searched were performed using PepSea™ software (Protana, Denmark) that was installed in-house against a upto date non-redundant protein database that was downloaded from the National Center for Biotechnology Information (NCBI) and against a database containing bacterial proteins.

The protein in sample WT2 was putatively identified as PG33, an immunoreactive 42 kDa antigen of P.gingivalis (NCBI accession no. AF175715.1 ;gi:5759279, deposited by Ross et al, 1998 and 1999; see Examples 32 and 33).

### Example 18

### Search Results Sample WT2

### Search (Auto 16) (WT2) Second ass

Entry name: Wild-type P.gingivalis strain W50, spot 2.

Average mass, pI: 43117.161 [Da,unmodified], 8.35.

DB Accession no.: gi:5759279.

Score: 237.

Matches: 10/55.

Total coverage; 123/385 (31.95%).

2RMS: 0.030 [Da] 20.920 [ppm].

Match Results

Masses are Protonated (MH+).

Cysteine is Carbamidomethyl-Cys. Methionine is Native

| Measured | Calculated | Mono | | | | | | |
|---|---|---|---|---|---|---|---|---|
| [Da] | [Da] | [Da] | [ppm] | Diff. | Diff. | Start | End Sequence | |
| 1131.674 | 1131.677 | Yes | −0.003 | −2.850 | 75 | 85 | (R)LSIVPTFGIGK(W) | [SEQ ID NO:14] |
| 1192.589 | 1192.553 | Yes | 0.036 | 30.196 | 86 | 94 | (K)WHEPYFGTR(L) | [SEQ ID NO:15] |
| 1209.701 | 1209.663 | Yes | 0.038 | 31.658 | 280 | 289 | (R)VVVDNVVYFR(J) | [SEQ ID NO:16] |
| 1493.719 | 1493.734 | Yes | −0.015 | −9.866 | 169 | 182 | (K)DDMTGTVNVGLMLK(F) | [SEQ ID NO:17] |
| 1656.729 | 1656.786 | Yes | −0.057 | −34.723 | 298 | 311 | (R)NQEINVYNTAEYAK(T) | [SEQ ID NO:18] |
| 1682.725 | 1682.741 | Yes | −0.016 | −9.219 | 364 | 377 | (K)GSSEQIYEENAWNR(I) | [SEQ ID NO:19] |
| 1804.854 | 1804.890 | Yes | −0.036 | −20.158 | 95 | 110 | (R)LQETGFDIYGFPQGSK(E) | [SEQ ID NO:20 |
| 2040.970 | 2040.999 | Yes | −0.028 | −13.878 | 295 | 311 | (K)IDRNQEINVYNTAEYAK(T) | [SEQ ID NO:21 |
| 2110.969 | 2111.001 | Yes | −0.032 | −15.326 | 262 | 279 | (R)RPVSCPECPEPTQPTVTR(V) | [SEQ ID NO:22] |

### Cysteine is Carbamidomethyl-Cys. Methionine is Oxidized

| Measured | Calculated | Mono | | | | | |
|---|---|---|---|---|---|---|---|
| [Da] | [Da] | [Da] | [ppm] | Diff. | Diff. | Start | End Sequence |
| 1613.793 | 1613.806 | Yes | −0.014 | −8.505 | 327 | 340 | (K)TGTAAYNMKLSERR(A) [SEQ ID NO:23] |

### Cysteine is Acrylamido-Cys. Methionine is Native

No peptides matched.

### Cysteine is Acrylamido-Cys. Methionine is Oxidized

No peptides matched.

### Example 19

### Amino Acid Sequence of Protein Identified as Highest Match with Peptides Obtained by Digestion of Excised Spot WT2.

MTYRIMKAKS LLLALAGLAC TFSATAQEAT TQNKAGMHTA
FQRDKASDHW FTDIAGGAGM ALSGWNNDVD FVDRLSIVPT
FGIGKWHEPY FGTRLOFTGF DIYGFPQGSK ERNHNYFGNA
HLDFMFDLTN YFGVYRPNRV FHIIPWAGIG FGYKFHSENA
NGEKVGSKDD MTGTVNVGLM LKFRLSRVVD FNIEGQAFAG
KMNFIGTKRG KADFPVMATA GLTFNLCKTE WTEIVPMDYA
LVNDLNNQIN SLRGQVEELS RRPVSCPECP EPTQPTVTRV
VVDNVVYFRI NSAKIDRNQE INVYNTAEYA KTNNAPIKVV
GYADEKTGTA AYNMKLSERR AKAVAKMLEK YGVSADRITI
EWKGSSEQIY EENAWNRIVV MTAAE

n.b. Peptides obtained by digestion of spot WT2 are underlined

### Example 20

### Nucleotide Sequence of Porphyromonas gingivalis Strain W50 Immunoreactive 42kD Antigen PG33 Gene (Accession no. AF175715.1; gi:5759278)

[SEQ ID NO:25]
5′-ATCAAAGCTAAATCTTTATTATTAGCACTTGCGGGTCTCGCATGCAC
ATTCAGTGCAACAGCCCAAGAAGCTACTACACAGAACAAGCAGGGATGCA
CACCGCATTCCAACGTGATAAGGCCTCCGATCATTGGTTCATTGACATTG
CAGGTGGAGCAGGTATGGCTCTCTCGGGATGGAATSSTGATGTAGACTTT
GTAGATCGTCTAAGTATCGTTCCTACTTTCGGTATCGGTAAATGGCATGA
GCCTTATTTCGGTACTCGTCTCCAATTCACAGGATTCGACATCTATGGAT
TCCCGCAAGGGAGCAAGGAGCGTAACCACAATTACTTTGGAAACGCCCAC
CTTGACTTCATGTTCGATCTGACGAACTATTTCGGTGTATACCGTCCCAA
TCGTGTCTTCCATATCATCCCATGGGCAGGTATAGGATTTGGTTATAAAT
TCCATAGCGAAAACGCCAATGGTGAAAAAGTAGGAAGTAAAGATGATATG
ACCGGAACAGTTAATGTCGGTTTGATGCTGAAATTCCGCCTATCAAGAGT
CGTAGACTTCAATATTGAAGGACAAGCTTTTGCCGGAAAGATGAACTTTA
TCGGGACAAAGAGAGGAAAAGCAGACTTCCCTGTAATGGCTACAGCAGGT
CTAACGTTCAACCTTGGCAAGACAGAGTGGACAGAAATTGTTCCTATGGA
CTATGCTTTGGTCAATGACCTGAACAACCAAATCAACTCACTTCGCCGTC
AAGTGGAAGAGTTGAGCCGTCGTCCTGTTTCATGCCCTGAATGCCCTGAG
CCTACACAGCCTACAGTTACTCGTGTAGTCGTTGACAATGTGGTTTACTT
CCGTATCAATAGTGCAAAGATTGATCGTAATCAAGAAATCAATGTTTACA
ATACAGCTGAATATGCGAAGACCAACAACGCACCGATCAAGGTAGTAGGT
TACGCTGACGAAAAAACCGGTACTGCGGCCTATAACATGAAGCTTTCAGA
GCGTCGTGCAAAAGCGGTAGCCAAGATGCTTGAAAAGTATGGTGTTTCTG
CGGATCGCATTACAATTGAATGGAAGGGCTCATCAGAGCAAATCTATGAA
GAGAACGCTTGGAATCGTATTGTAGTAATGACTGCAGCGGAATAA-3′

### Example 21

### Amino Acid Sequence of Porphyromonas gingivalis strain W50 immunoreactive 42 kD antigen PG33 gene (Accession no. AF175715.1; gi:5759278)

[SEQ ID NO:26]
NH₂-MKAKSLLLALAGLACTFSATAQEATTQNKAGMHTAFQRDKASDHWF
IDIAGGAGMALSGWNNDVDFVDRLSIVPTFGIGKWHEPYFGTRLQFTGFD
IYGFPQGSKERNHNYFGNAHLDFMFDLTNYFGVYRPNRVFHIIPWAGIGF
GYKFHSENANGEKVGSKDDMTGTVNCGLMLKFRLSRVVDFNIEGQAFAGK
MNFIGTKRGKADFPVMATAGLTFNLGKTEWTEIVPMDYALVNDLNNQINS
LRGQVEELSRRPVSCPECPEPTQPTVTRVVVDNVVYFRINSAKIDRNQEI
NVYNTAEYAKTNNAPIKVVGYADEKTGTAAYNMKLSERRAKAVAKMLEKY
GVSADRITIEWKGSSEQIYEENAWNRIVVMTAAE-COOH

### Example 22

### Alignment (Clustal Method) of the Amino Acid Sequences of Spot WT2/ sb/pog|344208 and the P.gingivalis P33 42 kDa antigen (NCBI AF175715.1;gi:5755279) Using the Megalign™ Algorithm, DNASTAR, Inc., Showing 100% Identity

| | M T Y R I M K A K S L L L A L A G L A C T F S A T A Q E A T | Majority |
|---|---|---|
| | \overline{ \| \| \| } | |
| | 10 20 30 | |
| | \| \| \| | |
| 1 | M T Y R I M K A K S L L L A L A G L A C T F S A T A Q E A T | WT2 Spot.pro |
| 1 | M - - - - - K A K S L L L A L A G L A C T F S A T A Q E A T | p33 Ag.pro |
| | T Q N K A G M H T A F Q R D K A S D H W F I D I A G G A G M | Majority |
| | \overline{ \| \| \| } | |
| | 40 50 60 | |
| | \| \| \| | |
| 31 | T Q N K A G M H T A F Q R D K A S D H W F I D I A G G A G M | WT2 Spot.pro |
| 26 | T Q N K A G M H T A F Q R D K A S D H W F I D I A G G A G M | p33 Ag.pro |
| | A L S G W N N D V D F V D R L S I V P T F G I G K W H E P Y | Majority |
| | \overline{ \| \| \| } | |
| | 70 80 90 | |
| | \| \| \| | |
| 61 | A L S G W N N D V D F V D R L S I V P T F G I G K W H E P Y | WT2 Spot.pro |
| 56 | A L S G W N N D V D F V D R L S I V P T F G I G K W H E P Y | ×Ag.pro |
| | F G T R L Q F T G F D I Y G F P Q G S K E R N H N Y F G N A | Majority |
| | \overline{ \| \| \| } | |
| | 100 110 120 | |
| | \| \| \| | |
| 91 | F G T R L Q F T G F D I Y G F P Q G S K E R N H N Y F G N A | WT2 Spot.pro |
| 86 | F G T R L Q F T G F D I Y G F P Q G S K E R N H N Y F G N A | p33 Ag.pro |
| | H L D F M F D L T N Y F G V Y R P N R V F H I I P W A G I G | Majority |
| | \overline{ \| \| \| } | |
| | 130 140 150 | |
| | \| \| \| | |
| 121 | H L D F M F D L T N Y F G V Y R P N R V F H I I P W A G I G | WT2 Spot.pro |
| 116 | H L D F M F D L T N Y F G V Y K P N R V F H I I P W A G I G | p33 Ag.pro |
| | F G Y K F H S E N A N G E K V G S K D D M T G T V N V G L M | Majority |
| | \overline{ \| \| \| } | |
| | 160 170 160 | |
| | \| \| \| | |
| 151 | F G Y K F H S E N A N G E K V G S K D D M T G T V N V G L M | WT2 Spot.pro |
| 146 | F G Y K F H S E N A N G E K V G S K D D M T G T V N V G L M | p33 Ag.pro |
| | L K F R L S R V V D F N I E G Q A F A G K M N F I G T K R G | Majority |
| | \overline{ \| \| \| } | |
| | 190 200 210 | |
| | \| \| \| | |
| 181 | L K F R L S R V V D F N I E G Q A F A G K M N F I G T K R G | WT2 Spot.pro |
| 176 | L K F R L S R V V D F N I E G Q A F A G K M N F I C T K R g | p33 Ag.pro |
| | K A D F P V M A T A G L T F N L G K T E W T E I V P M D T A | Majority |
| | \overline{ \| \| \| } | |
| | 220 230 240 | |
| | \| \| \| | |
| 211 | K A D F P V M A T A G L T F N L G K T E W T E I V P M D Y A | WT2 Spot.pro |
| 206 | K A D F P V M A T A G L T F N L G K T E W T E I V P M D Y A | p33 Ag.pro |
| | L V N D L N N Q I N S L R G V E E L S R R P V S C P E C P | Majority |
| | \overline{ \| \| \| } | |
| | 250 260 270 | |
| | \| \| \| | |
| 241 | L V N D L N N Q I N S L R G Q V E E L S R R P V S C P E C P | WT2 Spot.pro |
| 236 | L V N D L N N Q I N S L R G Q V E E L S R K P V S C P E C P | p33 Ag.pro |
| | E P T Q P T V T R V V V D N V V YV F R I N S A K I D R N Q E | Majority |
| | \overline{ \| \| \| } | |
| | 280 290 300 | |
| | \| \| \| | |
| 271 | E P T Q P T V T R V V V D N V V Y F R I N S A K I D R N Q E | WT2 Spot.pro |
| 266 | E P T Q P T V T K V V V D N V V Y F R I N S A K I D R N Q E | p33 Ag.pro |
| | I N V Y N T A E Y A K T N N A P I K V V G Y A D E K T G T A | Majority |
| | \overline{ \| \| \| } | |
| | 310 320 330 | |
| | \| \| \| | |
| 301 | I N V Y N T A E Y A K T N N A P I K V V G Y A D E K T G T A | WT2 Spot.pro |
| 296 | I N V Y N T A E Y A K T N N A P I K V V G Y A D E K T G T A | p33 Ag.pro |
| | A Y N M K L S E R R A K A V A K M L E K Y G V S A D R I T I | Majority |
| | \overline{ \| \| \| } | |
| | 340 350 360 | |
| | \| \| \| | |
| 331 | A Y N M K L S E R R A K A V A K M L E K Y G V S A D R I T I | WT2 Spot.pro |
| 326 | A Y N M K L S E R R A K A V A K M L E K Y G V S A D R I T I | p33 Ag pro |
| | E W K G S S E Q I Y E E N A W N R I V V M T A A E | Majority |
| | \overline{ \| \|} | |
| | 370 380 | |
| | \| \| | |
| 361 | E W K G S S E Q I Y E E N A W N R I V V M T A A E | WT2 Spot.pro |
| 356 | E W K G S S E Q I Y E E N A W N R I V V M T A A E | p33 Ag.pro |

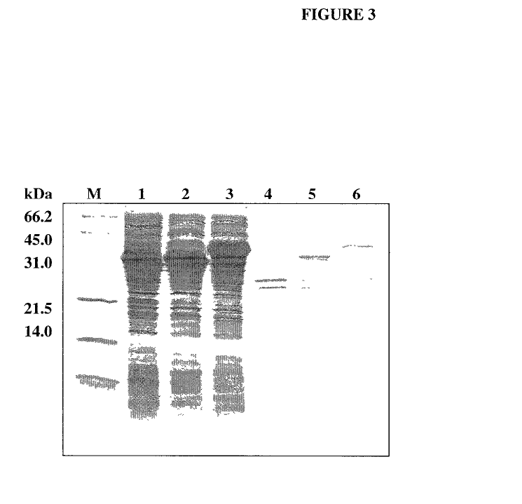

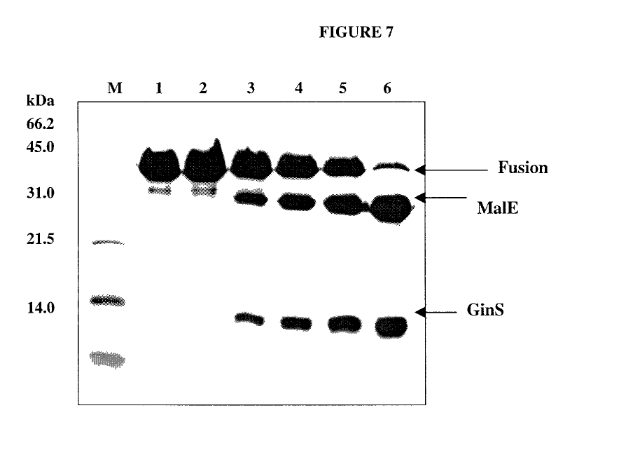

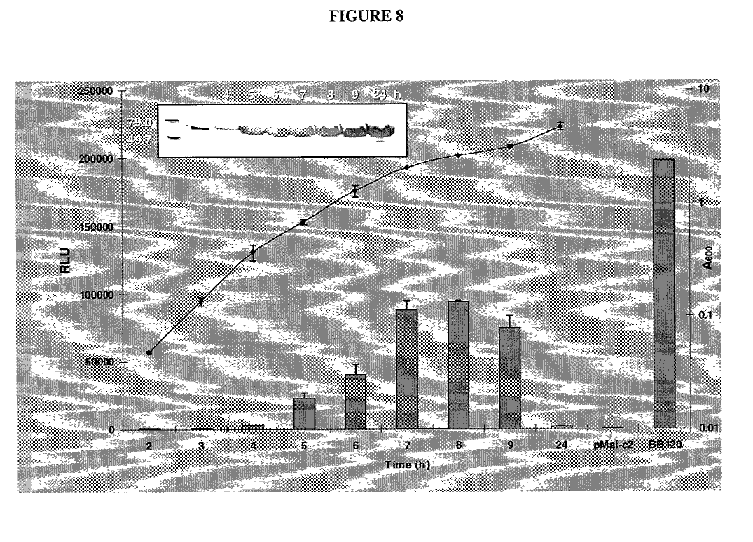

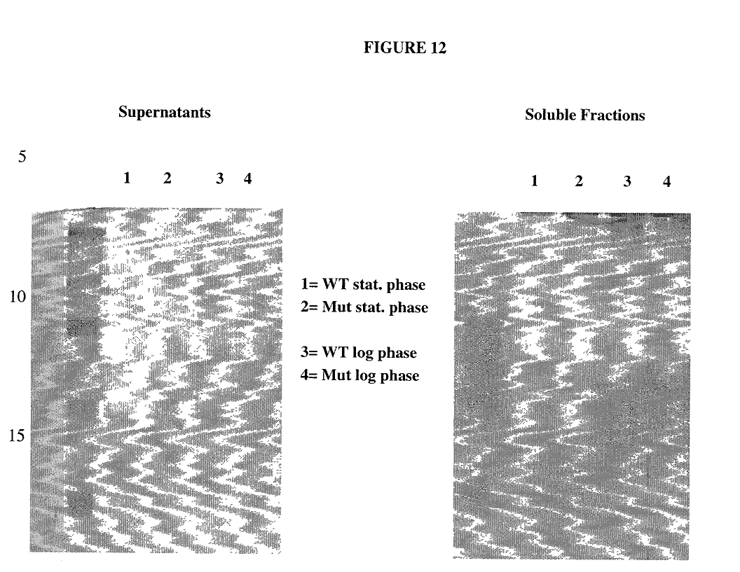

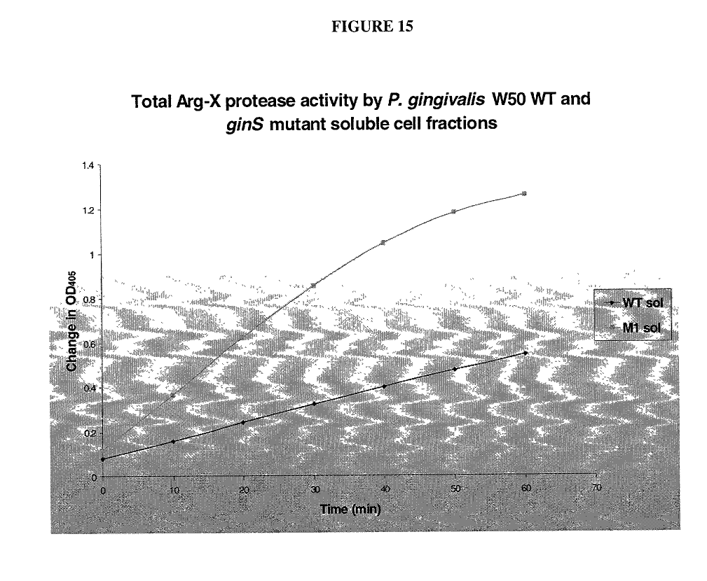

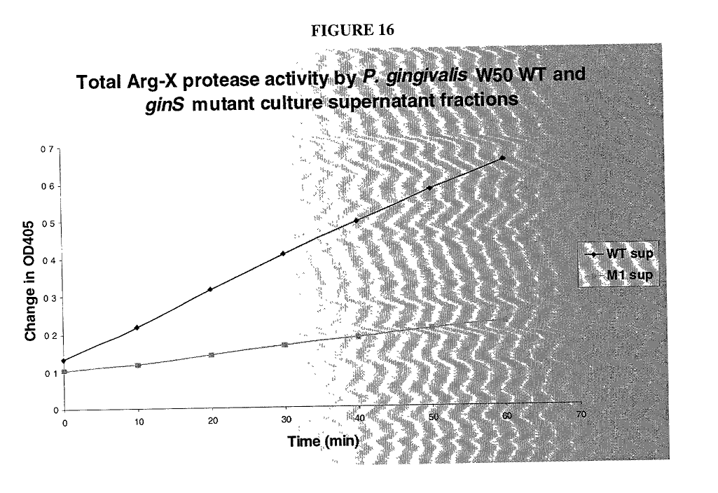

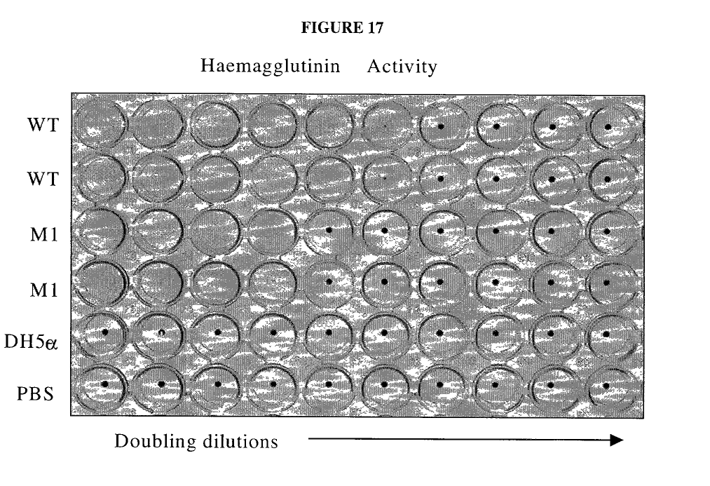

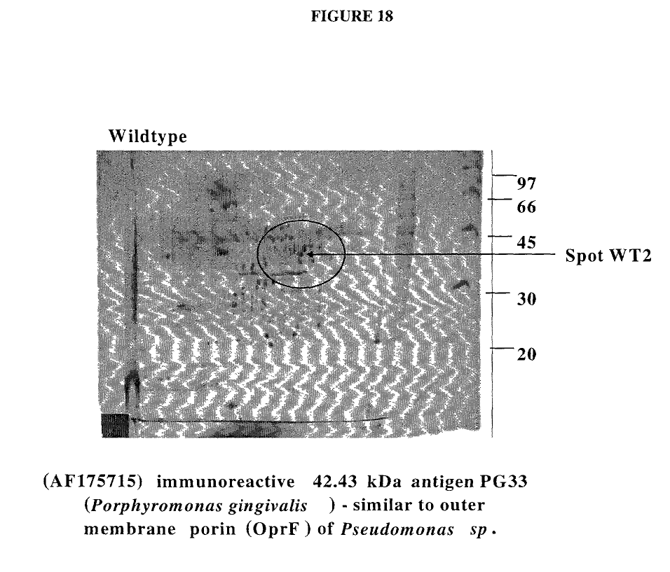

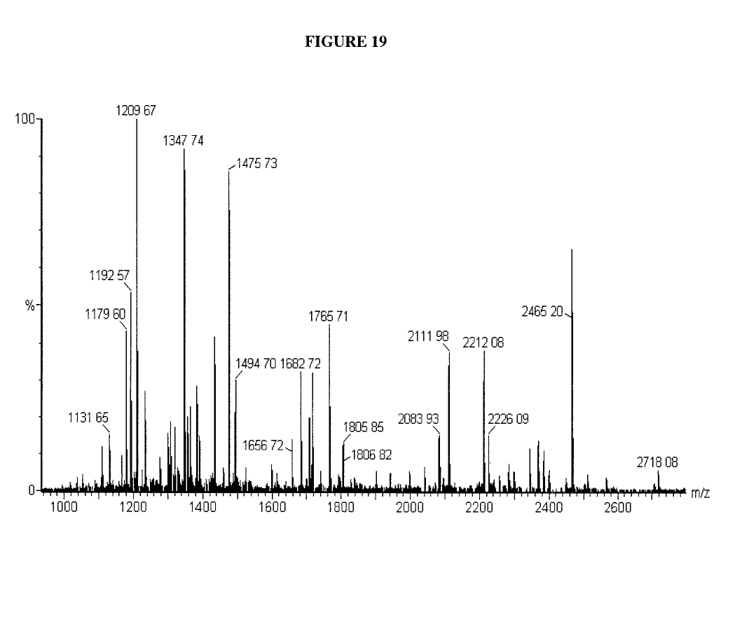

Discussion of Previously Described Data (see Examples 1-22 and FIGS. 1-19)

Quorum sensing describes a bacterial signalling mechanism, whereby, accumulation of molecules, known as autoinducers, allows individual bacteria to sense their environment and respond by regulating gene expression. Many Gram-negative bacteria employ a range of N-acyl-L-homoserine lactone (AHL) molecules as their signals and regulate expression of phenotypes, such as bioluminescence, using homologues of the V.fischeri LuxR/I proteins (Williams et al, FEMS Micro. Lett. 100: 161-168 (1992). However, a number of bacteria, including E.coli, B.subtilis and H.pylori, produce a molecule known as AI-2, the synthesis of which is driven by a homologue of the Vharveyi LuxS protein (Surette, et al., Proc. Natl. Acad. Sci, USA. 96: 1639-1644 (1999a). Forsyth, et al., Infect. Imman. 68: 3193-3199 (2000); Joyce et al, J. Bacteriol. 182: 3638-3643 (2000). AI-2 appears to represent a new family of signal molecules and it has been suggested that it is involved in cross-communication of bacteria because of its presence in both Gram-negatives and Gram-positives (Bassler et al,. J. Bacteriol. 179: 4043-4045 (1997)). In this study we have shown that P.gingivalis possesses a functional LuxS homologue, which we have designated GinS. This is the first evidence of a quorum sensing system in an anaerobic human pathogen. LuxS controls bioluminescence in V.harveyi (Surette et al, Proc. Natl. Acad. Sci, USA. 96: 1639-1644 (1999a)) and type III secretion in enterohemorrhagic (EHEC) and enteropathogenic (EPEC) E.coli, (Sperandio et al, PNAS 96: 15196-15201 (1999). A P.gingivalis ginS⁻ null mutant shows down-regulation of the major extracellular cysteine proteases, Rgp and Kgp, in culture supernatant in addition to a 4-fold decrease in haemagglutination of erythrocytes. These findings suggest that GinS plays a major role in the control of these important virulence factors. This finding is consistent with previous reports that cysteine proteases and haemagglutinin activities of P.gingivalis appear to be structurally related (Pavloff, et al., J. Biol. Chem. 270: 1007-1010 (1995).; Yoneda, et al., Genetic evidence for the relationship of Porphyromonas gingivalis cysteine protease and hemagglutinin activities. Oral Microbiol Immunol. 11: 129-134 (1996).

GinS bears low amino-acid homology with LuxS in E.coli. However, overproduction of the protein in E.coli DH50α and subsequent screening spent culture supernatant against V.harveyi sensor BB 170, complemented AI-2 production. This verifies that ginS encodes a related molecule to that of the other LuxS homologues. Detection of the signal molecule in P.gingivalis was achieved by growing the bacterium in a chemically defined medium and screening spent supernatants against BB170. The molecule is produced at mid-exponential growth and depleted by early stationary phase, which is consistant with results obtained from E.coli harbouring GinS. Western blot analysis of production of GinS in P.gingivalis and E.coli throughout the growth curve showed that the protein persists into late stationary phase, whereas the autoinducer depletes. It is predicted in E.coli and S.typhimitrium that there is an AI-2 degradation pathway (Surette and Bassler, 1998; Surette et al, 1999), therefore, it is interesting that the protein is not similarly lost. This perhaps suggests that the protein persists for direct activity under certain conditions and may differ in vivo, or it may have a secondary role as yet uncharacterised.

Using established biosensors and construction and analysis of a P.gingivalis W50 chromosomal library, no AHL molecules or LuxR/I homologues were identified. In addition, a search of the P.gingivalis genome sequence database provided further evidence that a V.fischeri system is not employed by this bacterium. However, because the genome sequence is still unfinished, these results are inconclusive.

We predict that GinS is active in P.gingivalis because of the regulation of protease expression and activity, as well as haemagglutinin activity, together with the ability of spent culture supernatants from P.gingivalis to induce luminescence in V.harveyi BB 170. Further work will involve determining if the molecule can restore protease and haemagglutinin activity in the ginS⁻ null mutant and using a number of techniques such as 2-D gel electrophoresis in attempt to isolate other associated targets.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

What is claimed is:

1. An isolated polypeptide selected from the group consisting of:
(i) an isolated polypeptide comprising an amino acid having at least 95% identity to the amino acid sequence of SEQ ID NO:2 over the entire length of SEQ ID NO:2;
(ii) an isolated polypeptide comprising the amino acid sequence of SEQ ID NO:2,
(iii) an isolated polypeptide that is the amino acid sequence of SEQ ID NO:2, and
(iv) a polypeptide that is encoded by a recombinant polynucleotide comprising the polyncleotide sequence of SEQ ID NO:1.

2. An isolated polynucleotide selected from the group consisting of:
(i) an isolated polynucleotide comprising a polynucleotide sequence encoding a polypeptide that has at least 95% identity to the amino acid sequence of SEQ ID NO:2, over the entire length of SEQ ID NO:2;
(ii) an isolated polynucleotide comprising a polynucleotide sequence that has at least 95% identity over its entire length to a polynucleotide sequence encoding the polypeptide of SEQ ID NO:2;
(iii) an isolated polynucleotide comprising a nucleotide sequence that has at least 95% identity to that of SEQ ID NO:1 over the entire length of SEQ ID NO:1;
(iv) an isolated polynucleotide comprising a nucleotide sequence encoding the polypeptide of SEQ ID NO:2;
(v) an isolated polynucleotide that is the polynucleotide of SEQ ID NO:1;
(vi) an isolated polynucleotide of at least 30 nucleotides in length obtainable by screening an appropriate library under stringent hybridization conditions with a probe having the sequence of SEQ ID NO:1 or a fragment thereof of of at least 30 nucleotides in length;
(vii) an isolated polynucleotide encoding a mature polypeptide expressed by the ginS gene comprised in the Porphyromonas gingivalis; and
(viii) a polynucleotide sequence complementary to said isolated polynucleotide of (i), (ii), (iii), (iv), (v), (vi) or (vii).

3. A method for the treatment of an individual:
(i) in need of enhanced activity or expression of or immunological response to the polypeptide of claim 1 comprising the step of: administering to the individual a therapeutically effective amount of an antagonist to said polypeptide; or
(ii) having need to inhibit activity or expression of the polypeptide of claim 1 comprising:
(a) administering to the individual a therapeutically effective amount of an antagonist to said polypeptide; or
(b) administering to the individual a nucleic acid molecule that inhibits the expression of a polynucleotide sequence encoding said polypeptide;
(c) administering to the individual a therapeutically effective amount of a polypeptide that competes with said polypeptide for its ligand, substrate, or receptor; or
(d) administering to the individual an amount of a polypeptide that induces an immunological response to said polypeptide in said individual.

4. A process for diagnosing or prognosing a disease or a susceptibility to a disease in an individual related to expression or activity of the polypeptide of claim 1 in an individual comprising the step of:
(a) determining the presence or absence of a mutation in the nucleotide sequence encoding said polypeptide in an organism in said individual; or
(b) analyzing for the presence or amount of said polypeptide expression in a sample derived from said individual.

5. A process for producing a polypeptide selected from the group consisting of:
(i) an isolated polypeptide comprising an amino acid sequence selected from the group having at least 95% identity to the amino acid sequence of SEQ ID NO:2 over the entire length of SEQ ID NO:2;
(ii) an isolated polypeptide comprising the amino acid sequence of SEQ ID NO:2;
(iii) an isolated polypeptide that is the amino acid sequence of SEQ ID NO:2, and
(iv) a polypeptide that is encoded by a recombinant polynucleotide comprising the polynucleotide sequence of SEQ ID NO:1, comprising the step of culturing a host cell under conditions sufficient for the production of the polypeptide.

6. A process for producing a host cell comprising an expression system or a membrane thereof expressing a polypeptide selected from the group consisting of:
(i) an isolated polypeptide comprising an amino acid sequence selected from the group having at least 95% identity to the amino acid sequence of SEQ ID NO:2 over the entire length of SEQ ID NO:2;
(ii) an isolated polypeptide comprising the amino acid sequence of SEQ ID NO:2;
(iii) an isolated polypeptide that is the amino acid sequence of SEQ ID NO:2, and
(iv) a polypeptide that is encoded by a recombinant polynucleotide comprising the polynucleotide sequence of SEQ ID NO:1, said process comprising the step of transforming or transfecting a cell with an expression system comprising a polynucleotide capable of producing said polypeptide of (i), (ii), (iii) or (iv) when said expression system is present in a compatible host cell such the host cell, under appropriate culture conditions, produces said polypeptide of (i), (ii), (iii) or (iv).

7. A host cell or a membrane expressing a polypeptide selected from the group consisting of:
(i) an isolated polypeptide comprising an amino acid sequence selected from the group having at least 95% identity to the amino acid sequence of SEQ ID NO:2 over the entire length of SEQ ID NO:2;
(ii) an isolated polypeptide comprising the amino acid sequence of SEQ ID NO:2;
(iii) an isolated polypeptide that is the amino acid sequence of SEQ ID NO:2, and
(iv) a polypeptide that is encoded by a recombinant polynucleotide comprising the polynucleotide sequence of SEQ ID NO:1.

8. An antibody immunospecific for the polypeptide of claim 1.

9. A method for screening to identify compounds that agonize or that inhibit the function of the polypeptide of claim 1 that comprises a method selected from the group consisting of:
(a) measuring the binding of a candidate compound to the polypeptide (or to the cells or membranes bearing the polypeptide) or a fusion protein thereof by means of a label directly or indirectly associated with the candidate compound;
(b) measuring the binding of a candidate compound to the polypeptide (or to the cells or membranes bearing the polypeptide) or a fusion protein thereof in the presence of a labeled competitor;
(c) testing whether the candidate compound results in a signal generated by activation or inhibition of the polypeptide, using detection systems appropriate to the cells or cell membranes bearing the polypeptide;
(d) mixing a candidate compound with a solution comprising a polypeptide of claim 1, to form a mixture, measuring activity of the polypeptide in the mixture, and comparing the activity of the mixture to a standard; or
(e) detecting the effect of a candidate compound on the production of mRNA encoding said polypeptide and said polypeptide in cells, using for instance, an ELISA assay.

10. An agonist or antagonist to the polypeptide of claim 1.
